(19) United States
(12) Patent Application Publication
(10) Pub. No.: US 2025/0313555 A1
(43) Pub. Date: Oct. 9, 2025

(54) 2-SUBSTITUTED THIAZOLE AND BENZOTHIAZOLE COMPOSITIONS AND METHODS AS DUX4 INHIBITORS

(71) Applicant: ALTAY THERAPEUTICS, INC., San Carlos, CA (US)

(72) Inventors: Ali OZES, San Carlos, CA (US); Lee LATIMER, Johnson City, TX (US)

(21) Appl. No.: 19/243,829

(22) Filed: Jun. 20, 2025

Related U.S. Application Data
(63) Continuation of application No. PCT/US2023/086566, filed on Dec. 30, 2023.
(60) Provisional application No. 63/436,362, filed on Dec. 30, 2022.
(60) Provisional application No. 63/616,498, filed on Dec. 29, 2023.

(51) Int. Cl. C07D 417/12; A61K 31/426; A61K 31/428; A61K 31/437; A61K 31/4439; A61K 31/444; A61K 31/506; C07D 277/30; C07D 277/32; C07D 277/46; C07D 277/66; C07D 277/82; C07D 417/04; C07D 417/14; C07D 513/04

(52) U.S. Cl. CPC C07D 417/12; A61K 31/426; A61K 31/428; A61K 31/437; A61K 31/4439; A61K 31/444; A61K 31/506; C07D 277/30; C07D 277/32; C07D 277/46; C07D 277/66; C07D 277/82; C07D 417/04; C07D 417/14; C07D 513/04

(57) ABSTRACT

Provided herein are compounds, compositions and methods for the treatment of a disease characterized by DUX4 misexpression. In certain embodiments, the inventive compounds and compositions can be administered either alone or in combination with other agents for a disease characterized by DUX4 misexpression.

### CROSS-REFERENCE

This application is Continuation of the International Patent Application No. PCT/US2023/086566, filed Dec. 30, 2023, which claims priority to, and the benefit of, U.S. Provisional Patent Application Nos. 63/436,362 (filed Dec. 30, 2022) and 63/616,498 (filed Dec. 29, 2023), which are incorporated herein by reference in their entirety.

### FIELD

Provided herein are 2-substituted thiazole and benzothiazole compounds, methods, and pharmaceutical compositions for use in treatment of diseases (e.g., neuromuscular disorders, inflammatory disorders, facioscapulohumeral muscular dystrophy, acute lymphoblastic leukemia, B-cell leukemia, sarcomas (e.g., small round cell sarcoma), prostate cancer, multiple myeloma, lung cancer, colon cancer, solid cancers, rheumatoid arthritis, axial spondyloarthitis, viral infections, mononucleosis, encephalitis, and varicella). In certain embodiments, 2-aryl thiazole or benzothiazole compounds are provided for the treatment of, for example, diseases characterized by double homeobox, 4 (DUX4) misexpression in a human, such as cancer.

### BACKGROUND OF THE INVENTION

The gene double homeobox, 4 (DUX4) is a gene of unknown function, the misregulation of which is responsible for, e.g., facioscapulohumeral muscular dystrophy. Lemmers, Richard J. L. F. et al., Science 2010, 329 (5999): 1650-3; doi: 10.1126/science.1189044. No current pharmaceutical method is known to control facioscapulohumeral muscular dystrophy effectively.

Therefore, there is a continuing need for compositions directed to effective treatment of diseases characterized by DUX4 misexpression and other diseases and conditions.

### SUMMARY OF THE INVENTION

Provided herein are compounds useful, for example, for treatment of diseases characterized by DUX4 misexpression (e.g., facioscapulohumeral muscular dystrophy; sarcoma; B-cell leukemia). In certain embodiments, the 2-aryl thiazole or benzothiazole compounds display remarkable efficacy or bioavailability, or both, in a human.

In certain embodiments, provided herein are compounds of Formula I:

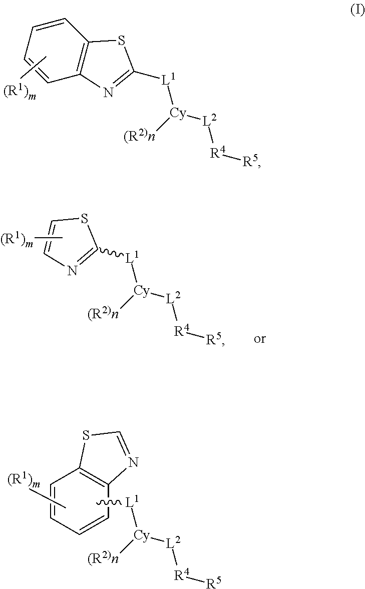

or a pharmaceutically acceptable salt thereof;
wherein:
   each R¹ is independently selected from the group including H and R²;
   m is an integer from 0 to 4;
   a wavy bond indicates a single bond attachment from L to a free site of a thiazole or benzothiazole ring,
   L¹ is selected from the group including a single bond, C₁₋₆alkyl, and —(C═O)—;
   L² is selected from the group including —(C═O)(NR³)—, —(C═O)-alkyl-, and —(NR³)(C═O)—;
   Cy is selected from the group including C₃₋₉cycloalkyl, C₃₋₉heterocycyl, C₃-C₉heteroaryl, and C₆₋₁₀aryl;
   each R² is independently selected from the group including halo, C₁₋₃alkoxy, C₁₋₃alkyl, cyano, and R⁵;
   n is an integer from 0 to 2;
   each R³ is independently selected from the group including H and C₁₋₃alkyl;
   R⁴ is C₁₋₆alkylene, wherein R⁴ is substituted with from 0 to 4 R⁷ groups;
   R⁵ is selected from the group consisting of —O(CO)R⁶, —N(R³)(CO)R³, —N(R³)(CO)R⁶, —OR⁶, —(CO)R⁶, —(CO)N(R³)R³, —(CO)N(R³)R⁶, C₃₋₇cycloalkyl, C₃₋₉heterocycyl, C₆₋₁₀aryl, and C₃-C₉heteroaryl;
   or, alternatively, R⁴ and R⁵ join to form a 5- to 8-membered cycloalkyl, aryl, heterocyclic, or heteroaryl ring that is optionally substituted with from 0 to 4 R⁷ groups;
   each R⁶ is independently selected from the group including C₁₋₆alkyl,
   C₃₋₇cycloalkyl, C₃₋₉heterocycyl, C₆₋₁₀aryl, and C₃-C₉heteroaryl; wherein R⁶ is substituted with from 0 to 4 R⁷ groups; and
   each R⁷ is independently selected from the group including halo, C₁₋₃alkoxy, and C₁₋₃alkyl.

In certain embodiments, provided herein are compounds of Formula II:

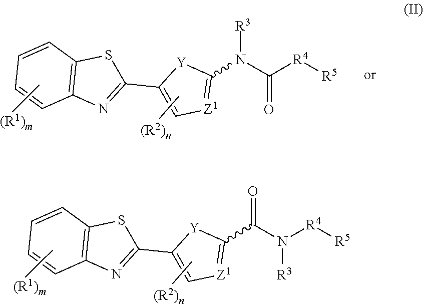

or a pharmaceutically acceptable salt thereof;
wherein:
   Y is selected from the group including CH, CR², —N═CH—, —N═CR²—, O, S, and N; wherein Y and Z¹ are not both N; and
   Z¹ is selected from the group including CH, CR², —N═CH—, —N═CR²—, and N; wherein Y and Z¹ are not both N.

In certain embodiments, provided herein are compounds, wherein the compounds are of Formula III:

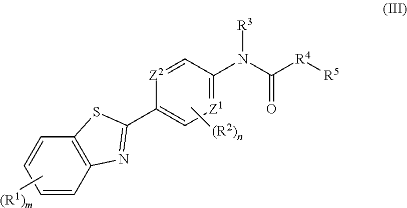

or a pharmaceutically acceptable salt thereof;
wherein:
   Z¹ and Z² are each selected from the group including CH, CR², and N; wherein Z¹ and Z² are not both N.

In certain embodiments, provided herein are compounds of Formula IV:

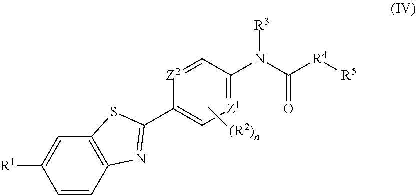

or a pharmaceutically acceptable salt thereof;
wherein:
   R¹ is selected from the group including H, halo, and C₁₋₃alkyl;
   each R² is independently selected from the group including halo, C₁₋₃alkoxy, and C₁₋₃alkyl;
   R⁴ is C₁₋₆alkylene;
   R⁵ is selected from the group including —O(CO)R⁶, —NH(CO)R⁶, —OR⁶, —(CO)R⁶, C₃₋₇cycloalkyl, and C₃₋₉heterocycyl; and
   R⁶ is selected from the group including C₁₋₆alkyl, C₃₋₇cycloalkyl, C₃₋₉heterocyclyl, and C₃₋₉heteroaryl.

In certain embodiments, Z¹ and Z² are each selected from the group including CH, CR², and N. In certain embodiments, Z¹ and Z² are each selected from the group including CH and N.

In certain embodiments, R¹ is H or methyl.

In certain embodiments, n is 0.

In certain embodiments, R⁴ is substituted with 0 R² groups.

In certain embodiments, R⁵ is selected from the group including —NH(CO)CH₃, —O(CO)CH₃, —(CO)CH₃, and —OCH₂CH₃.

In certain embodiments, the compound is selected from the group including the compounds of Table 6-1.

In certain embodiments, the compound inhibits the production of MBD3L2 RNA at 11 mM (e.g., per the procedure of Example 7).

In certain embodiments, the compound has a DUX4 EC₅₀ of <10 mM. In certain embodiments, the compound has a DUX4 EC₅₀ of <1 mM.

In certain embodiments, provided herein are pharmaceutical compositions comprising:
the compound as otherwise disclosed herein, and
a pharmaceutically acceptable excipient, carrier, or diluent.

In certain embodiments, the composition is an oral formulation.

In certain embodiments, provided herein are methods for the treatment of a patient comprising the administration of an effective amount of a compound or composition as otherwise disclosed herein. In certain embodiments, the patient is a human.

### DETAILED DESCRIPTION

### Description of Exemplary Embodiments

Provided herein are compounds, compositions and methods useful for treating cancer in a subject. Further provided are dosage forms useful for such methods.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In case of a plurality of definitions for a term herein, those in this section prevail unless stated otherwise.

All publications, patent applications, patents, and other references mentioned herein are incorporated herein by reference in their entirety. In case of conflict, the present application will control.

The articles “a,” “an,” and “the” as used herein not only include certain embodiments with a single member, but also may include embodiments with more than one member. For example, an aspect “comprising a compound of Formula Ib and an excipient” should be understood as presenting certain embodiments with at least a second compound of Formula Ib, at least a second excipient, or both.

Similarly, the term “or” as used herein is a Boolean “or,” unless the alternatives cannot be combined without logical incompatibility. For example, an aspect “comprising an excipient selected from A, B, or C” should be understood as applying to embodiments comprising A and B; B and C; A and C; or A, B, and C.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and to provide written description support for a claim limitation of, e.g., “0.98X.” When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The term “alkyl” as used herein, and unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁₋₁₀ alkyl. In certain embodiments, the alkyl group is C₁₋₁₂ alkyl; C₁₋₈ alkyl; or C₁₋₆ alkyl. In certain embodiments, the alkyl group is selected from the group including methyl, CF₃, CCl₃, CFCl₂, CF₂Cl, ethyl, CH₂CF₃, CF₂CF₃, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. The term includes both substituted and unsubstituted alkyl groups, including halogenated alkyl groups. In certain embodiments, the alkyl group is a fluorinated alkyl group. In certain embodiments, the alkyl group is unsubstituted. Non-limiting examples of moieties with which the alkyl group can be substituted are selected from the group including halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, cither unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl” as used herein, and unless otherwise specified, refers to a saturated straight or branched hydrocarbon having one to six carbon atoms, i.e., C₁ to C₆ alkyl. In certain embodiments, the lower alkyl group is a primary, secondary, or tertiary hydrocarbon. The term includes both substituted and unsubstituted moieties. In certain embodiments, the lower alkyl group is unsubstituted.

The term “alkylene” as used herein, and unless otherwise specified, refers to divalent saturated aliphatic hydrocarbon groups (particularly having from one to eleven carbon atoms) which can be straight-chained or branched. In certain embodiments, the alkylene group contains 1 to 6 carbon atoms. The term includes both substituted and unsubstituted moieties. In certain embodiments, the alkylene group is unsubstituted. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “alkenyl” as used herein, and unless otherwise specified, refers to monovalent olefinically unsaturated hydrocarbon groups, in certain embodiments, having up to about 11 carbon atoms, from 2 to 8 carbon atoms, or from 2 to 6 carbon atoms, which can be straight-chained or branched and having at least 1 or from 1 to 2 sites of olefinic unsaturation. The term includes both substituted and unsubstituted moieties. In certain embodiments, the alkenyl group is unsubstituted. Exemplary alkenyl groups include ethenyl (i.e., vinyl, or —CH═CH₂), n-propenyl (—CH₂CH═CH₂), isopropenyl (—C(CH₃)═CH₂), and the like.

The term “alkenylene” as used herein, and unless otherwise specified, refers to divalent olefinically unsaturated hydrocarbon groups, in certain embodiments, having up to about 11 carbon atoms or from 2 to 6 carbon atoms which can be straight-chained or branched and having at least 1 or from 1 to 2 sites of olefinic unsaturation. The term includes both substituted and unsubstituted moieties. In certain embodiments, the alkenylene group is unsubstituted. This term is exemplified by groups such as ethenylene (—CH═CH—), the propenylene isomers (e.g., —CH═CHCH₂—; —C(CH₃)═CH—; and —CH═C(CH₃)—), and the like.

The term “alkynyl” as used herein, and unless otherwise specified, refers to acetylenically unsaturated hydrocarbon groups, in certain embodiments, having up to about 11 carbon atoms or from 2 to 6 carbon atoms which can be straight-chained or branched and having at least 1 or from 1 to 2 sites of alkynyl unsaturation. The term includes both substituted and unsubstituted moieties. In certain embodiments, the alkynyl group is unsubstituted. Non-limiting examples of alkynyl groups include acetylenic, ethynyl (—C≡CH), propargyl (—CH₂C≡CH), and the like.

The term “alkoxy” as used herein, and unless otherwise specified, refers to the group —OR′ in which R′ is alkyl or cycloalkyl. Alkoxy groups include, for example, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxycarbonyl” as used herein, and unless otherwise specified, refers to a radical —C(O)-alkoxy. Alkoxycarbonyl groups include, for example, methoxycarbonyl, ethoxycarbonyl, tert-butoxycarbonyl, and the like.

The term “aryl” as used herein, and unless otherwise specified, refers to phenyl, biphenyl, or naphthyl. The term includes both substituted and unsubstituted moieties. In certain embodiments, an aryl group can be substituted with any described moiety, including, but not limited to, one or more moieties selected from the group consisting of halogen (fluoro, chloro, bromo or iodo), alkyl, haloalkyl, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, the entire contents of which are herein incorporated by reference in its entirety for all purposes.

In certain embodiments, an aryl group refers to a phenyl or naphthyl group optionally mono- or disubstituted by a fluoro, chloro, bromo, iodo, cyano, trifluoromethyl, nitro, carboxy, aminocarbonyl, C₁₋₃-alkyl (i.e., a one- to three-carbon alkyl group), or C₁₋₃-alkoxy group. In certain embodiments, the aryl group is unsubstituted.

The term “amino” as used herein, and unless otherwise specified, refers to the radical —NH₂.

The term “monoalkylamino” as used herein, and unless otherwise specified, refers to the group alkyl-NR′—, wherein R′ is selected from hydrogen and alkyl or cycloalkyl.

The term “alkylamino” or “arylamino” as used herein, and unless otherwise specified, refers to an amino group that has one or two alkyl or aryl substituents, respectively. In certain embodiments, the alkyl substituent is lower alkyl. In certain embodiments, the alkyl or lower alkyl is unsubstituted.

The term “acyl” as used herein, and unless otherwise specified, refers to a group of the formula C(O)R′, wherein R′ is alkyl or cycloalkyl (including lower alkyl) (i.e., “alkanoyl”), aryl including phenyl, alkaryl, arylalkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or arylalkyl sulphonyl including methanesulfonyl, the mono, di or triphosphate ester, trityl or monomethoxy-trityl, substituted benzyl, alkaryl, arylalkyl including benzyl, alkoxyalkyl including methoxymethyl, aryloxyalkyl such as phenoxymethyl. Aryl groups in the esters comprise a phenyl group. In particular, acyl groups include acetyl, trifluoroacetyl, methylacetyl, cyclpropylacetyl, propionyl, butyryl, hexanoyl, heptanoyl, octanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzencacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, and 4-phenylbenzoyl.

The term “carboxyl” or “carboxy” as used herein, and unless otherwise specified, refers to the radical —C(O)OH.

The term “cycloalkyl,” as used herein, and unless otherwise specified, refers to a saturated cyclic hydrocarbon. In certain embodiments, the cycloalkyl group may be saturated, bridged or non-bridged, and/or a fused bicyclic group. In certain embodiments, the cycloalkyl group includes three to ten carbon atoms, i.e., C₃ to C₁₀ cycloalkyl. In some embodiments, the cycloalkyl has from 3 to 15 (C₃₋₁₅), from 3 to 10 (C₃₋₁₀), or from 3 to 7 (C₃₋₇) carbon atoms. In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. The term includes both substituted and unsubstituted moieties. In certain embodiments, the cycloalkyl group is unsubstituted.

The term “cycloalkenyl,” as used herein, and unless otherwise specified, refers to an unsaturated cyclic hydrocarbon. In certain embodiments, cycloalkenyl refers to mono- or multicyclic ring systems that include at least one double bond. In certain embodiments, the cycloalkenyl group may be a bridged, non-bridged, and/or a fused bicyclic group. In certain embodiments, the cycloalkyl group includes three to ten carbon atoms, i.e., C₃ to C₁₀ cycloalkyl. In some embodiments, the cycloalkenyl has from 3 to 7 (C₃₋₇), or from 4 to 7 (C₄₋₇) carbon atoms. The term includes both substituted and unsubstituted moieties. In certain embodiments, the cycloalkenyl group is unsubstituted.

The term “halogen” or “halo” as used herein, and unless otherwise specified, refers to chloro, bromo, fluoro or iodo.

The term “heterocyclyl” or “heterocyclic” as used herein, and unless otherwise specified, refers to a monovalent monocyclic non-aromatic ring system or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Heterocyclyl groups are bonded to the rest of the molecule through the non-aromatic ring. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, pipcridinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. The term includes both substituted and unsubstituted moieties. In certain embodiments, the heterocyclyl group is unsubstituted.

The term “heteroaryl” as used herein, and unless otherwise specified, refers to refers to a monovalent monocyclic aromatic group and/or multicyclic aromatic group that contain at least one aromatic ring, wherein at least one aromatic ring contains one or more heteroatoms independently selected from O, S, and N in the ring. Heteroaryl groups are bonded to the rest of the molecule through the aromatic ring. Each ring of a heteroaryl group can contain up to one or two O atoms, one or two S atoms, or one to four N atoms, provided that the total number of ring heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. The term includes both substituted and unsubstituted moieties. In certain embodiments, the heteroaryl group is unsubstituted.

The term “protecting group” as used herein, and unless otherwise specified, refers to a group that is added to an oxygen, nitrogen, or phosphorus atom to prevent its further reaction or for other purposes. A wide variety of oxygen and nitrogen protecting groups are known to those skilled in the art of organic synthesis.

The term “pharmaceutically acceptable salt” as used herein, and unless otherwise specified, refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

The term “substantially free of” or “substantially in the absence of” as used herein, and unless otherwise specified, with respect to a composition refers to a composition that includes at least 85 or 90% by weight, in certain embodiments 95%, 98%, 99%, or 100% by weight, of the designated enantiomer of that compound. In certain embodiments, in the methods and compounds provided herein, the compounds are substantially free of other enantiomers or diastereomers.

Similarly, the term “isolated” as used herein, and unless otherwise specified, with respect to a composition refers to a composition that includes at least 85%, 90%, 95%, 98%, 99% to 100% by weight, of the compound, the remainder comprising other chemical species or enantiomers.

The term “solvate” as used herein, and unless otherwise specified, refers to a compound provided herein or a salt thereof, that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “cycloalkyl,” “alkenyl,” “cycloalkenyl,” “alkynyl,” “aryl,” “alkoxy,” “alkoxycarbonyl,” “carboxyl,” “alkylamino,” “arylamino,” “heterocyclyl,” “heteroaryl,” “acyl,” and “carboxyl” groups optionally comprise deuterium at one or more positions where hydrogen atoms are present, and wherein the deuterium composition of the atom or atoms is other than the natural isotopic composition.

Also as used herein, “alkyl,” “cycloalkyl,” “alkenyl,” “cycloalkenyl,” “alkynyl,” “aryl,” “alkoxy,” “alkoxycarbonyl,” “carboxyl,” “alkylamino,” “arylamino,” “heterocyclyl,” “heterocyclic,” “heteroaryl,” “acyl,” and “carboxyl” groups optionally comprise carbon-13 at an amount other than the natural isotopic composition.

As used herein, the term “EC₅₀” refers to a dosage, concentration, or amount of a test compound that elicits a dose-dependent response at 50% of maximal expression of a particular response that is induced, provoked or potentiated by the test compound.

As used herein, the term “IC₅₀” refers to an amount, concentration, or dosage of a particular test compound that achieves a 50% inhibition of a maximal response in an assay that measures such response.

The terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is refractory or non-responsive to current treatments for a proliferative disease. In another embodiment, the subject is a farm animal (e.g., a horse, a cow, a pig, etc.) or a pet (e.g., a dog or a cat). In certain embodiments, the subject is a human.

The terms “therapeutic agent” and “therapeutic agents” as used herein, and unless otherwise specified, refer to any agent(s) which can be used in the treatment of a disorder or one or more symptoms thereof. In certain embodiments, the term “therapeutic agent” includes a compound provided herein. In certain embodiments, a therapeutic agent is an agent which is known to be useful for, has been, or is currently being used for the treatment of a disorder or one or more symptoms thereof.

The term “therapeutically effective amount” or “effective amount” as used herein, and unless otherwise specified, refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In certain embodiments, “treating” or “treatment” includes modulating the disease or disorder, either physically (e.g., stabilization of a discernible symptom) or physiologically (e.g., stabilization of a physical parameter) or both. In certain embodiments, “treating” or “treatment” includes delaying the progression of the disease or disorder.

As used herein, the terms “prophylactic agent” and “prophylactic agents” refer to any agent(s) which can be used in the prevention of a disorder or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, has been, or is currently being used to prevent or impede the onset, development, progression and/or severity of a disorder.

As used herein, the phrase “prophylactically effective amount” refers to the amount of a therapy (e.g., prophylactic agent) which is sufficient to result in the prevention or reduction of the development, recurrence, or onset of one or more symptoms associated with a disorder, or to enhance or to improve the prophylactic effect(s) of another therapy (e.g., another prophylactic agent).

### Compounds

In certain embodiments, provided herein are compounds of Formula I:

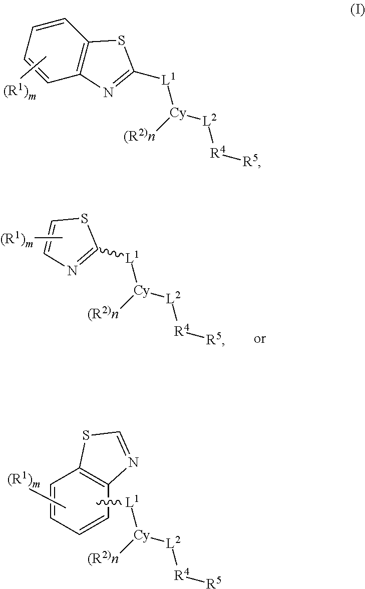

or a pharmaceutically acceptable salt thereof;
wherein:
   each R¹ is independently selected from the group including H and R²;
   m is an integer from 0 to 4;
   a wavy bond indicates a single bond attachment from L to a free site of a thiazole or benzothiazole ring,
   L¹ is selected from the group including a single bond, C₁₋₆alkyl, and —(C═O)—;
   L² is selected from the group including —(C═O)(NR³)—, —(C═O)-alkyl-, and (NR³)(C═O)—;
   Cy is selected from the group including C₃₋₉cycloalkyl, C₃₋₉heterocycyl, C₃-C₉heteroaryl, and C₆₋₁₀aryl;
   each R² is independently selected from the group including halo, C₁₋₃alkoxy, C₁₋₃alkyl, cyano, and R⁵;
   n is an integer from 0 to 2;
   each R³ is independently selected from the group including H and C₁₋₃alkyl;
   R⁴ is C₁₋₆alkylene, wherein R⁴ is substituted with from 0 to 4 R⁷ groups;
   R⁵ is selected from the group consisting of —O(CO)R⁶, —N(R³)(CO)R³, —N(R³)(CO)R⁶, —OR⁶, —(CO)R⁶, —(CO)N(R³)R³, —(CO)N(R³)R⁶, C₃₋₇cycloalkyl, C₃₋₉heterocycyl, C₆₋₁₀aryl, and C₃-C₉heteroaryl;
   or, alternatively, R⁴ and R⁵ join to form a 5- to 8-membered cycloalkyl, aryl, heterocyclic, or heteroaryl ring that is optionally substituted with from 0 to 4 R⁷ groups;
   each R⁶ is independently selected from the group including C₁₋₆alkyl, C₃₋₇cycloalkyl, C₃₋₉heterocycyl, C₆₋₁₀aryl, and C₃-C₉heteroaryl; wherein R⁶ is substituted with from 0 to 4 R⁷ groups; and
   each R⁷ is independently selected from the group including halo, C₁₋₃alkoxy, and C₁₋₃alkyl.

In certain embodiments, provided herein are compounds of Formula I-B:

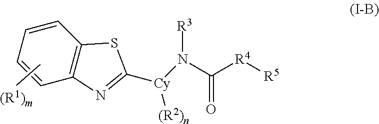

or a pharmaceutically acceptable salt thereof;
wherein:
   each R¹ is independently selected from the group including H and R²;
   m is an integer from 0 to 4;
   Cy is selected from the group including C₃₋₉heterocycyl, C₃-C₉heteroaryl, and C₆₋₁₀aryl;
   each R² is independently selected from the group including halo, C₁₋₃alkoxy, C₁₋₃alkyl, and R⁵;
   n is an integer from 0 to 2;
   R³ is selected from the group including H and C₁₋₃alkyl;
   R⁴ is C₁₋₆alkylene, wherein R⁴ is substituted with from 0 to 4 R⁷ groups;
   each R⁵ is independently selected from the group including —O(CO)R⁶, —NH(CO)R⁶, —OR⁶, —(CO)R⁶, C₃₋₇cycloalkyl, C₃₋₉heterocycyl, C₆₋₁₀aryl, and C₃-C₉heteroaryl;
   each R⁶ is independently selected from the group including C₁₋₆alkyl,
   C₃₋₇cycloalkyl, C₃₋₉heterocycyl, C₆₋₁₀aryl, and C₃-C₉heteroaryl; wherein R⁶ is substituted with from 0 to 4 R⁷ groups; and
   each R⁷ is independently selected from the group including halo, C₁₋₃alkoxy, and C₁₋₃alkyl.

In certain embodiments, provided herein are compounds of Formula II:

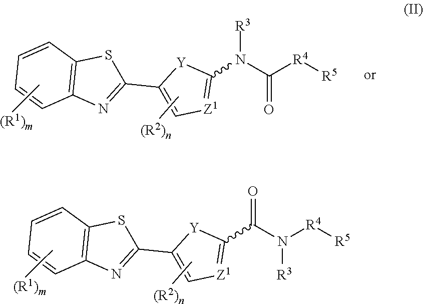

or a pharmaceutically acceptable salt thereof;
wherein:
   Y is selected from the group including CH, CR², —N═CH—, —N═CR²—, O, S, and N;
   wherein Y and Z¹ are not both N; and
   Z¹ is selected from the group including CH, CR², —N═CH—, —N—CR²—, and N; wherein Y and Z¹ are not both N.

In certain embodiments, provided herein are compounds of Formula II-B:

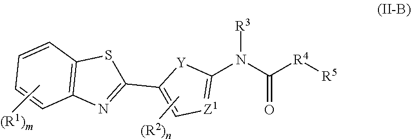

or a pharmaceutically acceptable salt thereof;
wherein:
   Y is selected from the group including CH, CR², O, S, and N; wherein Y and Z¹ are not both N; and
   Z¹ is selected from the group including CH, CR², and N; wherein Y and Z¹ are not both N.

In certain embodiments, Y is O and Z¹ is N, CH, or CR². In certain embodiments, Y is O and Z¹ is CH or CR² (e.g., CH). In certain embodiments, Y is O and Z¹ is N.

In certain embodiments, provided herein are compounds, wherein the compounds are of Formula III:

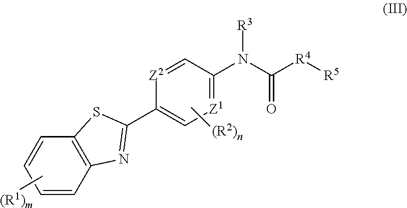

or a pharmaceutically acceptable salt thereof;
wherein:
   Z¹ and Z² are each selected from the group including CH, CR², and N; wherein Z¹ and Z² are not both N.

In certain embodiments, Z¹ is N and Z² is CH or CR² (e.g., CH). In certain embodiments, Z¹ is CH or CR² (e.g., CH) and Z² is N. In certain embodiments, Z¹ and Z² are both independently selected from CH or CR². In certain embodiments, Z¹ and Z² are both CH. In certain embodiments, Z¹ and Z² are each selected from the group including CH and N.

In certain embodiments, provided herein are compounds of Formula IV:

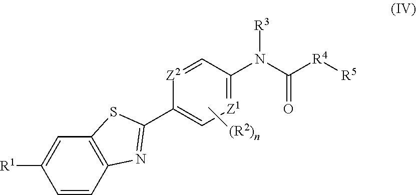

or a pharmaceutically acceptable salt thereof;
wherein:
   R¹ is selected from the group including H, halo, and C₁₋₃alkyl;
   each R² is independently selected from the group including halo, C₁₋₃alkoxy, and C₁₋₃alkyl;
   R⁴ is C₁₋₆alkylene;
   R⁵ is selected from the group including —O(CO)R⁶, —NH(CO)R⁶, —OR⁶, —(CO)R⁶, C₃₋₇cycloalkyl, and C₃₋₉heterocycyl; and
   R⁶ is selected from the group including C₁₋₆alkyl, C₃₋₇cycloalkyl, C₃₋₉heterocyclyl, and C₃₋₉heteroaryl.

In certain embodiments, R¹ is independently selected from the group including H, F, Cl, methoxy, methyl, ethyl, and acetoxy. In certain embodiments, R¹ is independently selected from the group including H and methyl.

In certain embodiments, R¹ is H or methyl.

In certain embodiments, R¹ is 6-substituted. In certain embodiments, R¹ is 5-substituted. In certain embodiments, R¹ is 7-substituted. In certain embodiments, R¹ is 4-substituted.

In certain embodiments, m is an integer from 0 to 4 (e.g., 0, 1, 2, 3, or 4). In certain embodiments, m is 0, 1, 2, or 3. In certain embodiments, m is 0, 1, or 2. In certain embodiments, m is 0 or 1. In certain embodiments, m is 0.

In certain embodiments, Cy is selected from the group including C₃₋₉heterocycyl, C₃-C₉heteroaryl, and C₆₋₁₀aryl. In certain embodiments, Cy is selected from the group including C₃-C₉heteroaryl and C₆₋₁₀aryl. In certain embodiments, Cy is C₆₋₁₀aryl (e.g., a phenyl ring). In certain embodiments, Cy is C₃-C₉heteroaryl (e.g., furanyl; pyridinyl; pyrimidinyl).

In certain embodiments, each R² is independently selected from the group including halo, C₁₋₃alkoxy, C₁₋₃alkyl, and R⁵. is independently selected from the group including H, F, Cl, methoxy, methyl, ethyl, and acetoxy. In certain embodiments, R¹ is independently selected from the group including H and methyl.

In certain embodiments, n is an integer from 0 to 2 (e.g., 0, 1, or 2). In certain embodiments, n is 0 or 1. In certain embodiments, n is 0.

In certain embodiments, each R³ is independently selected from the group including H and C₁₋₃alkyl. In certain embodiments, R³ is H. In certain embodiments, R³ is methyl. In certain embodiments, R³ is ethyl.

In certain embodiments, R⁴ is C₁₋₆alkylene, wherein R⁴ is substituted with from 0 to 0.4 R⁷ groups. In certain embodiments, R⁴ is methylene. In certain embodiments, R⁴ is ethylene, propylene, or butylene. In certain embodiments, R⁴ is substituted with 0 R⁷ groups. In certain embodiments, R⁴ is substituted with 1 or 2 0 R⁷ groups (e.g., methyl).

In certain embodiments, R⁴ is substituted with 0 R² groups.

In certain embodiments, R⁵ is selected from the group including —O(CO)R⁶, —N(R³)(CO)R³, —N(R³)(CO)R⁶, —OR⁶, —(CO)R⁶, —(CO)N(R³)R³, —(CO)N(R³)R⁶, C₃₋₇cycloalkyl, C₃₋₉heterocycyl, C₆₋₁₀aryl, and C₃-C₉heteroaryl. In certain embodiments, R⁵ is selected from the group including —O(CO)R⁶, —N(R³)(CO)R⁶, —OR⁶, —(CO)R⁶, —(CO)N(R³)R⁶, C₃₋₇cycloalkyl, C₃₋₉heterocycyl, C₆₋₁₀aryl, and C₃-C₉heteroaryl.

In certain embodiments, R⁵ is independently selected from the group including —O(CO)R⁶, —NH(CO)R⁶, —OR⁶, —(CO)R⁶, C₃₋₇cycloalkyl, C₃₋₉heterocycyl, C₆₋₁₀aryl, and C₃-C₉heteroaryl. In certain embodiments, R⁵ is —O(CO)R⁶ (e.g., acetoxy). In certain embodiments, R⁵ is —NH(CO)R⁶ (e.g., acetamido). In certain embodiments, R⁵ is —OR⁶ (e.g., methoxy, ethoxy, or isopropoxy). In certain embodiments, R⁵ is —(CO)R⁶ (e.g., —(CO)Me). In certain embodiments, R⁵ is C₃₋₇cycloalkyl (e.g., cyclopropyl, cyclobutyl, cyclopentyl, or cyclohexyl). In certain embodiments, R⁵ is C₃₋₉heterocycyl (e.g., 1-substituted pyrrolidine-2,5-dione; 1-substituted pyrrolidin-2-one; 5-substituted pyrrolidin-2-one). In certain embodiments, R⁵ is C₆₋₁₀aryl (e.g., phenyl; naphthyl). In certain embodiments, R⁵ is C₃-C₉heteroaryl (e.g., 1-, 2-, 4-, or 5-imidazolyl; 1- or 4-triazolyl; 1-, 3-, 4-, or 5-pyrazolyl; 2-, 4-, or 5-oxazolyl; 2-, 3-, or 4-pyridyl; 1-, 3-, 4-, 5-, or 6-substituted pyridin-2-one; 2-, 4-, 5-, or 6-pyrimidinyl).

In certain embodiments, R⁵ is selected from the group including —NH(CO)CH₃, —O(CO)CH₃, —(CO)CH₃, and —OCH₂CH₃.

In certain embodiments, each R⁶ is independently selected from the group including C₁₋₆alkyl (e.g., methyl, ethyl, or isopropyl), C₃₋₇cycloalkyl, C₃₋₉heterocycyl, C₆₋₁₀aryl, and C₃-C₉heteroaryl; wherein R⁶ is substituted with from 0 to 4 R⁷ groups. In certain embodiments, R⁶ is C₁₋₆alkyl (e.g., methyl, ethyl, or isopropyl). In certain embodiments, R⁶ is C₃₋₇cycloalkyl (e.g., cyclopropyl, cyclobutyl, cyclopentyl, or cyclohexyl). In certain embodiments, R⁶ is C₃₋₉heterocycyl (e.g., 1-substituted pyrrolidine-2,5-dione; 1-substituted pyrrolidin-2-one; 5-substituted pyrrolidin-2-one). In certain embodiments, R⁶ is C₆₋₁₀aryl (e.g., phenyl; naphthyl). In certain embodiments, R⁶ is C₃-C₉heteroaryl (e.g., 1-, 2-, 4-, or 5-imidazolyl; 1- or 4-triazolyl; 1-, 3-, 4-, or 5-pyrazolyl; 2-, 4-, or 5-oxazolyl; 2-, 3-, or 4-pyridyl; 1-, 3-, 4-, 5-, or 6-substituted pyridin-2-one; 2-, 4-, 5-, or 6-pyrimidinyl).

In certain embodiments, R⁶ is substituted with from 0 to 4 independently selected R⁷ groups. In certain embodiments, R⁶ is substituted with from 0 to 3 independently selected R⁷ groups. In certain embodiments, R⁶ is substituted with from 0 to 2 independently selected R⁷ groups. In certain embodiments, R⁶ is substituted with from 0 to 1 R⁷ groups. In certain embodiments, R⁶ is substituted with no R⁷ groups.

In certain embodiments, each R⁷ is independently selected from the group including halo, C₁₋₃alkoxy, and C₁₋₃alkyl. In certain embodiments, each R⁷ is independently selected from the group including halo and C₁₋₃alkyl. In certain embodiments, each R⁷ is halo (e.g., F). In certain embodiments, each R⁷ is C₁₋₃alkyl (e.g., methyl). In certain embodiments, each R⁷ is C₁₋₃alkoxy (e.g., methoxy; ethoxy).

In certain embodiments, provided herein is a compound selected from the group including:

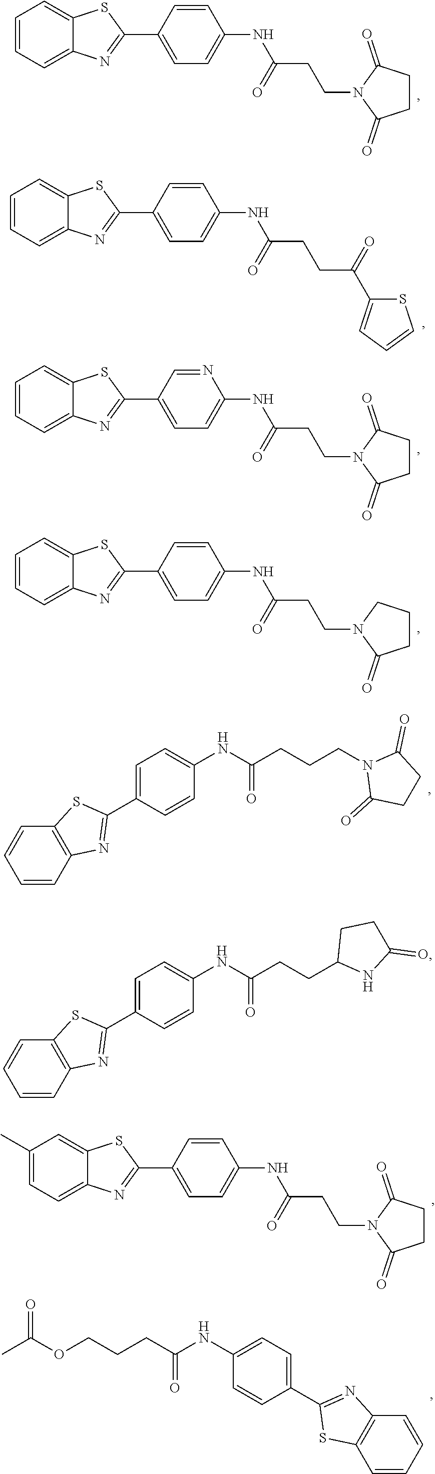

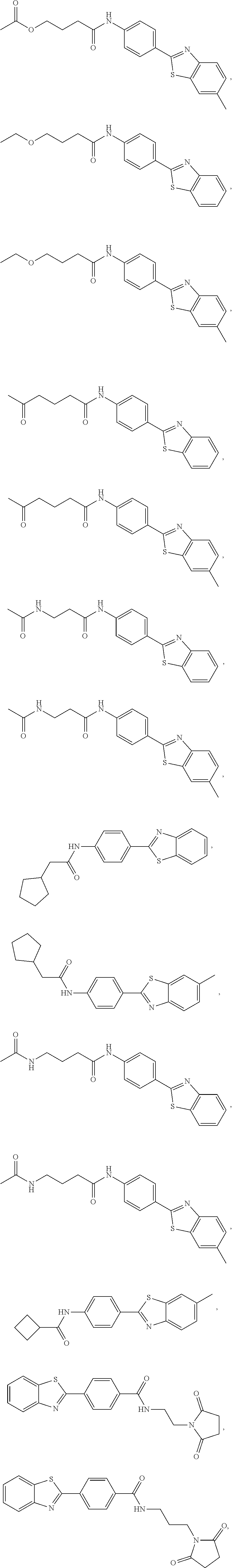

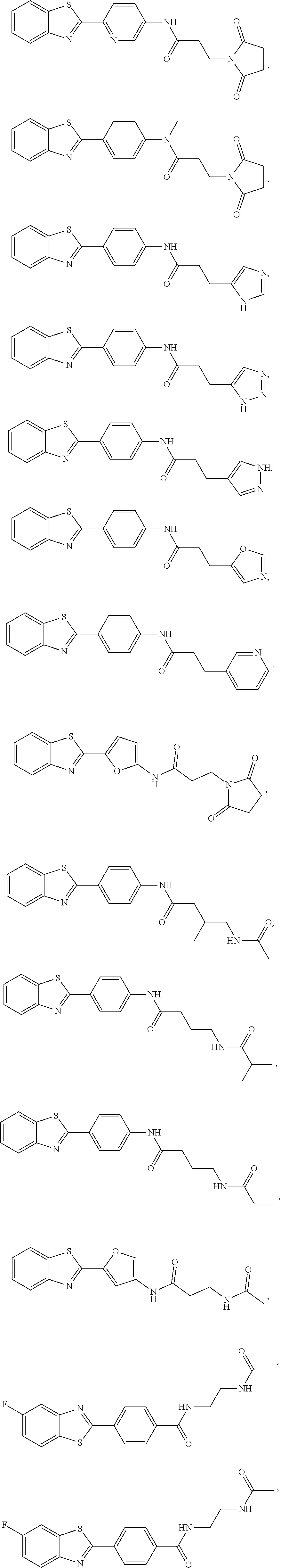

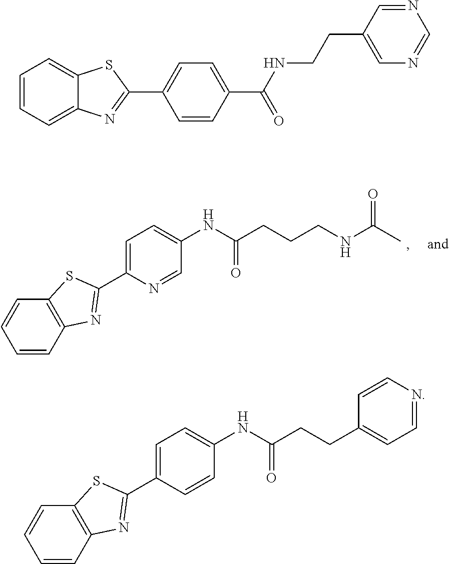 [SMILES: O=C(NCCc1cncnc1)c1ccc(-c2nc3ccccc3s2)cc1 ; CC(=O)NCCCC(=O)Nc1ccc(-c2nc3ccccc3s2)nc1 ; O=C(CCc1ccncc1)Nc1ccc(-c2nc3ccccc3s2)cc1]

In certain embodiments, provided herein is a compound selected from the group including:

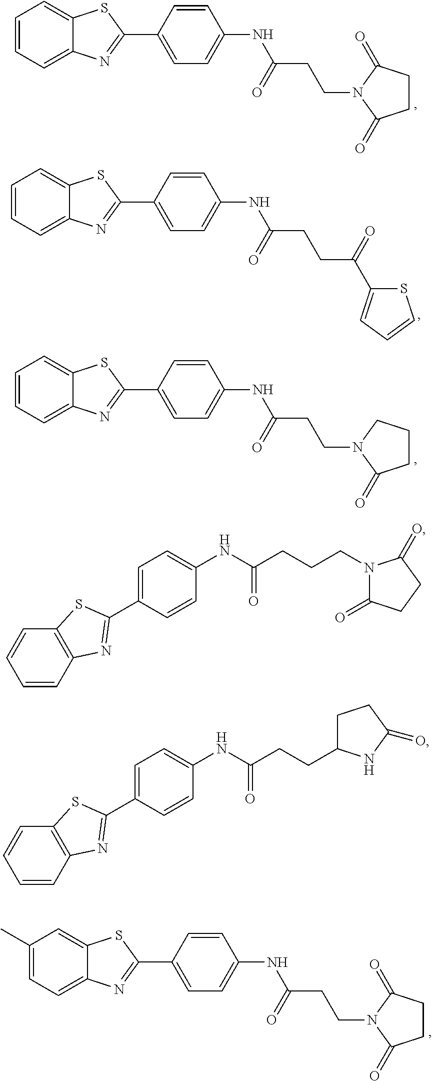

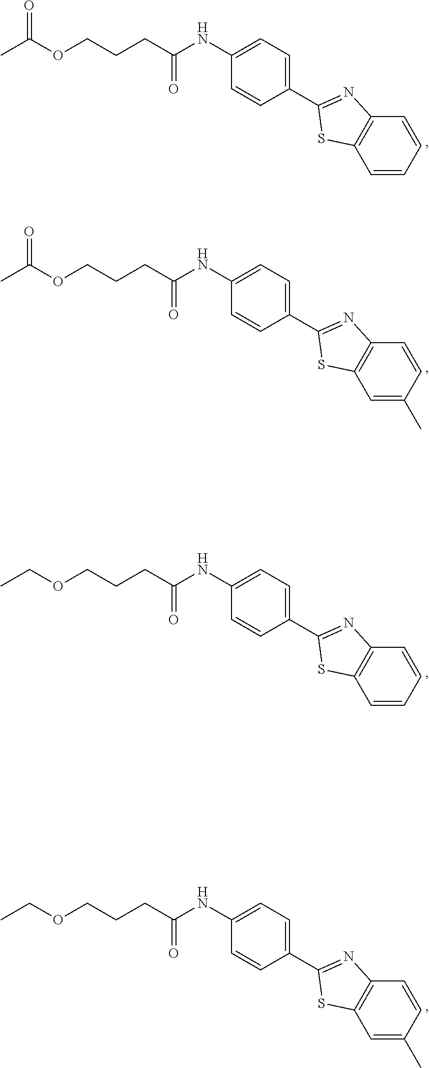

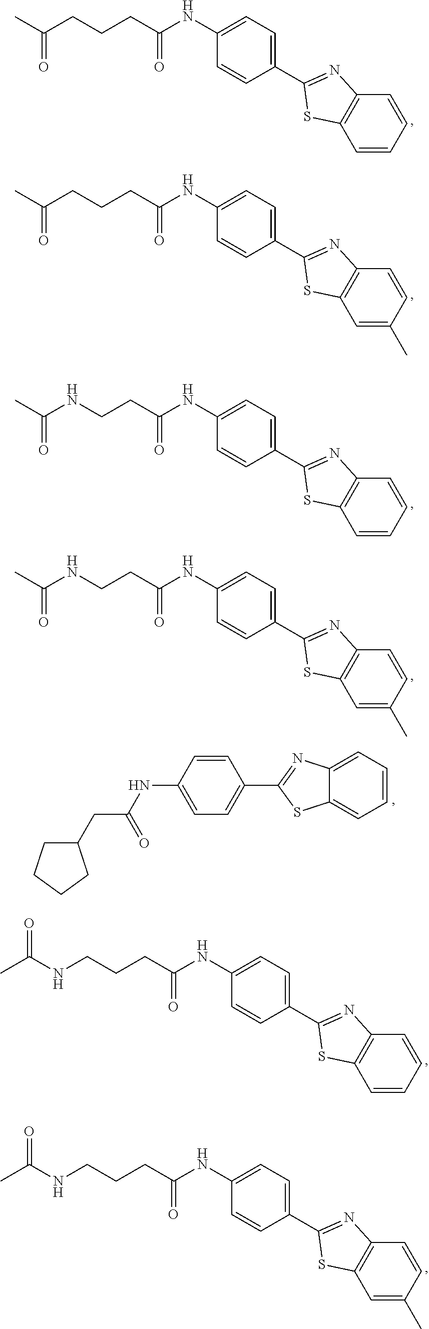

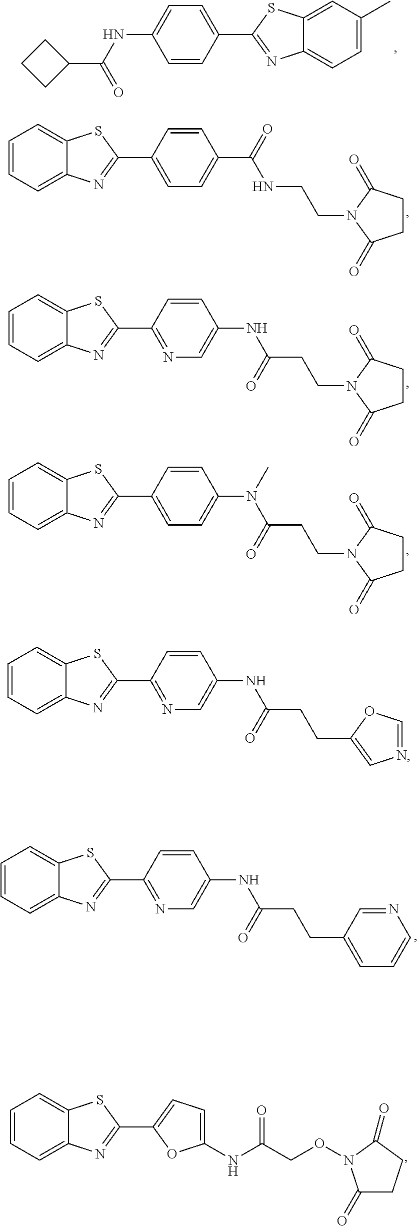

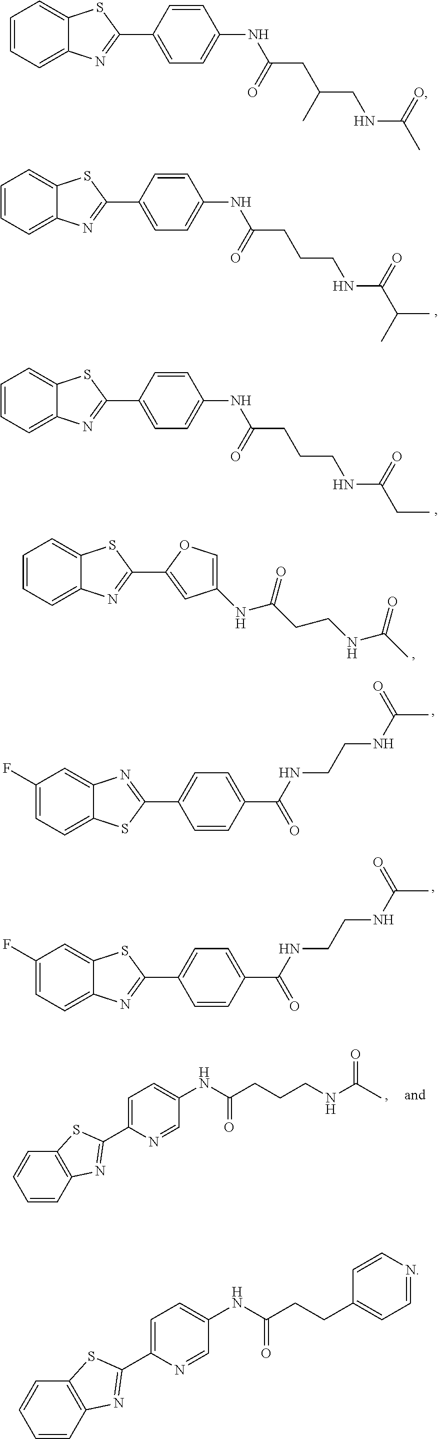

In certain embodiments, the compound is selected from the group including the compounds of Table 6-1. In certain embodiments, the compound is any one compound of Table 6-1.

In certain embodiments, one or more variables of Formula (I) or of Formula (I-B) (i.e., R¹, m, Cy, R², n, R³, R⁴, R⁵, R⁶, or R⁷) correspond to that of a compound of Table 6-1.

In certain embodiments, one or more variables of Formula (II) or of Formula (II-B) (i.e., R¹, m, Y, Z¹, R², n, R³, R⁴, R⁵, R⁶, or R⁷) correspond to that of a compound of Table 6-1.

In certain embodiments, one or more variables of Formula (III) (i.e., R¹, m, Z¹, Z², R², n, R³, R⁴, R⁵, R⁶, or R⁷) correspond to that of a compound of Table 6-1.

In certain embodiments, one or more variables of Formula (IV) (i.e., R¹, Z¹, Z², R², n, R³, R⁴, R⁵, R⁶, or R⁷) correspond to that of a compound of Table 6-1.

In certain embodiments, the compound inhibits the production of MBD3L2 RNA at 11 mM (e.g., per the procedure of Example 7).

In certain embodiments, the compound has a DUX4 EC₅₀ of <10 mM (e.g., coded as “B” or “C” in Table 6-1). In certain embodiments, the compound has a DUX4 EC₅₀ of <1 mM (e.g., coded as “C” in Table 6-1).

In some embodiments, provided herein are:
(a) compounds as described herein, e.g., of Formula I, II, III, or IV, and pharmaceutically acceptable salts and compositions thereof;
(b) compounds as described herein, e.g., of Formula I, II, III, or IV, and pharmaceutically acceptable salts and compositions thereof for use in the treatment and/or prophylaxis of diseases characterized by DUX4 misexpression;
(c) processes for the preparation of compounds as described herein, e.g., of Formula I, II, III, or IV, as described in more detail elsewhere herein;
(d) pharmaceutical formulations comprising a compound as described herein, e.g., of Formula I, II, III, or IV, or a pharmaceutically acceptable salt thereof together with a pharmaceutically acceptable carrier or diluent; and
(e) pharmaceutical formulations comprising a compound as described herein, e.g., of Formula I, II, III, or IV, or a pharmaceutically acceptable salt thereof together with one or more other effective pharmaceutical agents for diseases characterized by DUX4 misexpression, optionally in a pharmaceutically acceptable carrier or diluent.

### Optically Active Compounds

The compounds provided herein may have several chiral centers and may exist in and be isolated in optically active and racemic forms. Some compounds may exhibit polymorphism. Any racemic, optically active, diastereomeric, polymorphic, or stereoisomeric form, or mixtures thereof, of a compound provided herein, which possess the useful properties described herein is within the scope of the invention. Preparation of optically active forms can be prepared by any methods known to the skilled artisan (for example, by resolution of the racemic form by recrystallization techniques, by synthesis from optically active starting materials, by chiral synthesis, or by chromatographic separation using a chiral stationary phase).

Examples of methods to obtain optically active materials are known in the art and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In some embodiments, compositions of the inventive compounds are substantially free of a designated enantiomer of that compound. In certain embodiments, in the methods and compounds of this invention, the compounds are substantially free of enantiomers. In some embodiments, the composition includes that includes a compound that is at least 85%, 90%, 95%, 98%, 99% to 100% by weight, of the compound, the remainder comprising other chemical species or enantiomers.

### Isotopically Enriched Compounds

Also provided herein are isotopically enriched compounds.

Isotopic enrichment (for example, deuteration) of pharmaceuticals to improve pharmacokinetics (“PK”), pharmacodynamics (“PD”), and toxicity profiles has been demonstrated previously with some classes of drugs. See, e.g., Lijinsky et al., Food Cosmet. Toxicol., 20:393 (1982); Lijinsky et al., J. Nat. Cancer Inst., 69:1127 (1982); Mangold et al., Mutation Res. 308:33 (1994); Gordon et al., Drug Metab. Dispos., 15:589 (1987); Zello et al., Metabolism, 43:487 (1994); Gately et al., J. Nucl. Med., 27:388 (1986); Wade D, Chem. Biol. Interact. 117:191 (1999).

Isotopic enrichment of a drug can be used, for example, (1) to reduce or eliminate unwanted metabolites, (2) to increase the half-life of the parent drug, (3) to decrease the number of doses needed to achieve a desired effect, (4) to decrease the amount of a dose necessary to achieve a desired effect, (5) to increase the formation of active metabolites, if any are formed, or (6) to decrease the production of deleterious metabolites in specific tissues or to create a more effective or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and it occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species, such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations before the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart greater or lesser toxicity.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### Preparation of Compounds

The compounds provided herein can be prepared, isolated or obtained by any method apparent to those of skill in the art. Exemplary methods of preparation are described in detail in the examples below.

### Pharmaceutical Compositions and Methods of Administration

In certain embodiments, provided herein are pharmaceutical compositions comprising
a compound as otherwise disclosed herein; and
a pharmaceutically acceptable excipient, carrier, or diluent.

In certain embodiments, the composition is an oral formulation.

In certain embodiments, the compounds can be formulated into pharmaceutical compositions using methods available in the art and those disclosed herein. Any of the compounds disclosed herein can be provided in the appropriate pharmaceutical composition and be administered by a suitable route of administration.

The methods provided herein encompass administering pharmaceutical compositions containing at least one compound as described herein, including a compound of general Formula I, II, III, or IV, if appropriate in the salt form, either used alone or in the form of a combination with one or more compatible and pharmaceutically acceptable carriers, such as diluents or adjuvants, or with another pharmaceutical agent for treating diseases characterized by DUX4 misexpression.

In certain embodiments, the second agent can be formulated or packaged with the compound provided herein. The second agent will only be formulated with the compound provided herein when, according to the judgment of those of skill in the art, such co-formulation should not interfere with the activity of either agent or the method of administration. In certain embodiments, the compound provided herein and the second agent are formulated separately. They can be packaged together, or packaged separately, for the convenience of the practitioner of skill in the art.

In clinical practice the active agents provided herein may be administered by any conventional route, such as orally, parenterally, rectally, or by inhalation (e.g., in the form of aerosols). In certain embodiments, the compound provided herein is administered orally.

Use may be made, as solid compositions for oral administration, of tablets, pills, hard gelatin capsules, powders, or granules. In these compositions, the active product is mixed with one or more inert diluents or adjuvants, such as sucrose, lactose, or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening, or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin, the entire contents of which are herein incorporated by reference in its entirety for all purposes.

Typical pharmaceutical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 2d. Ed., Marcel Dekker, NY, NY, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically scaled foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in certain embodiments, in purified form, together with a suitable amount of carrier so as to provide the form for proper administration to the subject. The formulation should suit the mode of administration. In a certain embodiment, the pharmaceutical compositions or single unit dosage forms are sterile and in suitable form for administration to a subject, for example, an animal subject, such as a mammalian subject, for example, a human subject.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to case pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing, Easton PA (2000).

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. In certain embodiments, a dosage form can have about 0.1, 0.2, 0.3, 0.4, 0.5, 1.0, 2.0, 2.5, 5.0, 10.0, 15.0, 20.0, 25.0, 50.0, 100, 200, 250, 500, or 1000 mg of the active compound.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton PA (2000).

In certain embodiments, the oral dosage forms are solid and prepared under anhydrous conditions with anhydrous ingredients, as described in detail in the sections above. However, the scope of the compositions provided herein extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their case of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant—that is, neither too much nor too little to detrimentally alter the release of the active ingredients—should be used to form solid oral dosage forms. The amount of disintegrant used varies based upon the type of formulation and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, MD), a coagulated aerosol of synthetic silica (marketed by Degussa Co. of Plano, TX), CAB O SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

### Delayed Release Dosage Forms

Active ingredients such as the compounds provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500; each of which is incorporated herein by reference in its entirety. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus, encompassed herein are single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In certain embodiments, a pump may be used (see, e.g., Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Transdermal, Topical & Mucosal Dosage Forms

Also provided are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. Sec, e.g., Remington's Pharmaceutical Sciences, 16^{th}, 18th and 20^{th} eds., Mack Publishing, Easton PA (1980, 1990 & 2000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period to permit the penetration of a desired amount of active ingredients.

Suitable excipients (e.g., carriers and diluents) and other materials that can be used to provide transdermal, topical, and mucosal dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are non toxic and pharmaceutically acceptable. Moisturizers or humectants can also be added to pharmaceutical compositions and dosage forms if desired. Examples of such additional ingredients are well known in the art. Sec, e.g., Remington's Pharmaceutical Sciences, 16^{th}, 18^{th} and 20^{th} eds., Mack Publishing, Easton PA (1980, 1990 & 2000).

Depending on the specific tissue to be treated, additional components may be used before, in conjunction with, or after treatment with active ingredients provided. For example, penetration enhancers can be used to assist in delivering the active ingredients to the tissue. Suitable penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates, or solvates of the active ingredients can be used to further adjust the properties of the resulting composition.

### Dosage and Unit Dosage Forms

In certain embodiments, provided herein are methods for the treatment of a patient comprising the administration of an effective treatment amount of a compound or composition as otherwise disclosed herein. In certain embodiments, the patient is a human.

In human therapeutics, the doctor will determine the posology which he considers most appropriate according to a preventive or curative treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

In further aspects, provided are methods of treating or preventing a disease characterized by DUX4 misexpression in a subject by administering to a subject in need thereof an effective amount of a compound provided herein, or a pharmaceutically acceptable salt thereof. The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a subject is 0.140 mg/kg to 3 mg/kg of the subject's body weight, based on weight of the active compound. In certain embodiments, the dosage administered to a subject is between 0.20 mg/kg and 2.00 mg/kg, or between 0.30 mg/kg and 1.50 mg/kg of the subject's body weight.

In certain embodiments, the recommended daily dose range of a composition provided herein for the conditions described herein lie within the range of from about 0.1 mg to about 1000 mg per day, given as a single once-a-day dose or as divided doses throughout a day. In certain embodiments, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above-described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiment, the dosage of the composition provided herein, based on weight of the active compound, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, treatment or prevention can be initiated with one or more loading doses of a compound or composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. In certain embodiments, each maintenance does is, independently, about from about 10 mg to about 200 mg per day, between about 25 mg and about 150 mg per day, or between about 25 and about 80 mg per day. Maintenance doses can be administered daily and can be administered as single doses, or as divided doses.

In certain embodiments, a dose of a compound or composition provided herein can be administered to achieve a steady-state concentration of the active ingredient in blood or serum of the subject. The steady-state concentration can be determined by measurement according to techniques available to those of skill or can be based on the physical characteristics of the subject such as height, weight and age. In certain embodiments, a sufficient amount of a compound or composition provided herein is administered to achieve a steady-state concentration in blood or serum of the subject of from about 300 to about 4000 ng/mL, from about 400 to about 1600 ng/ml, or from about 600 to about 1200 ng/mL. In some embodiments, loading doses can be administered to achieve steady-state blood or serum concentrations of about 1200 to about 8000 ng/ml, or about 2000 to about 4000 ng/ml for one to five days. In certain embodiments, maintenance doses can be administered to achieve a steady-state concentration in blood or serum of the subject of from about 300 to about 4000 ng/ml, from about 400 to about 1600 ng/ml, or from about 600 to about 1200 ng/mL.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated, while the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising a compound, or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In certain embodiments, the unit dosages comprise about 1, 5, 10, 25, 50, 100, 125, 250, 500 or 1000 mg active ingredient. Such unit dosages can be prepared according to techniques familiar to those of skill in the art.

The dosages of the second agents are to be used in the combination therapies provided herein. In certain embodiments, dosages lower than those which have been or are currently being used to prevent or treat a disease characterized by DUX4 misexpression are used in the combination therapies provided herein. The recommended dosages of second agents can be obtained from the knowledge of those of skill. For those second agents that are approved for clinical use, recommended dosages are described in, for example, Hardman et al., eds., 1996, Goodman & Gilman's The Pharmacological Basis Of Therapeutics 9^{th} Ed, Mc-Graw-Hill, New York; Physician's Desk Reference (PDR) 57^{th} Ed., 2003, Medical Economics Co., Inc., Montvale, NJ, which are incorporated herein by reference in their entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week part, at about 1 to 2 weeks apart, or more than 2 weeks apart.

In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, a compound provided herein and a second agent are administered to a patient, for example, a mammal, such as a human, in a sequence and within a time interval such that the compound provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In certain embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In certain embodiments, a compound as provided herein and a second agent are cyclically administered to a patient. Cycling therapy involves the administration of a first agent (e.g., a first prophylactic or therapeutic agents) for a period, followed by the administration of a second agent and/or third agent (e.g., a second and/or third prophylactic or therapeutic agents) for a second period and by repeating this sequential administration. Cycling therapy can reduce the development of resistance to one or more of the therapies, avoid or reduce the side effects of one of the therapies, and/or improve the efficacy of the treatment.

In certain embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In certain embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered before or after administration of a second agent. Also contemplated are administration of a compound provided herein and a second agent by the same or different routes of administration, e.g., oral and parenteral. In certain embodiments, when the compound provided herein is administered concurrently with a second agent that potentially produces adverse side effects including, but not limited to, toxicity, the second active agent can advantageously be administered at a dose that falls below the threshold that the adverse side effect is elicited.

### Kits

Also provided are kits for use in methods of treatment of diseases characterized by DUX4 misexpression. The kits can include a compound or composition provided herein, a second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Methods of Use

In certain embodiments, provided herein are methods for the treatment of a patient comprising the administration of an effective treatment amount of a compound or composition as otherwise disclosed herein. In certain embodiments, the patient is a human. In certain embodiments, the patient is a subject in need of the treatment (i.e., a patient in need thereof). In certain embodiments, the patent is a subject who was previously treated with another chemotherapeutic compound or composition.

In certain embodiments, provided herein are methods for the treatment and/or prophylaxis of diseases characterized by DUX4 misexpression that includes the administration of an effective amount of a compound provided herein, or a pharmaceutically acceptable salt thereof. In certain embodiments, provided herein are methods for treating a disease characterized by DUX4 misexpression in a subject. In certain embodiments, the methods encompass the step of administering to the subject in need thereof an amount of a compound effective for the treatment or prevention of a disease characterized by DUX4 misexpression in combination with a second agent effective for the treatment or prevention of the disease. The compound can be any compound as described herein, and the second agent can be any second agent described in the art or herein. In certain embodiments, the compound is in the form of a pharmaceutical composition or dosage form, as described elsewhere herein.

In certain embodiments, the subject has never received therapy or prophylaxis for a disease characterized by DUX4 misexpression. In further embodiments, the subject has previously received therapy or prophylaxis for a disease characterized by DUX4 misexpression.

In certain embodiments, the subject is a subject that discontinued a therapy for the disease characterized by DUX4 misexpression because of one or more adverse events associated with the therapy. In certain embodiments, the subject is a subject where current therapy is not indicated.

In certain embodiments, the subject has received a therapy for a disease characterized by DUX4 misexpression and has discontinued that therapy before administration of a method provided herein. In further embodiments, the subject has received therapy and continues to receive that therapy along with administration of a method provided herein. The methods can be co-administered with other therapy for the disease according to the judgment of one of skill in the art. In certain embodiments, the methods or compositions provided herein can be co-administered with a reduced dose of the other therapy for the disease characterized by DUX4 misexpression.

In certain embodiments, provided are methods of treating a subject that is refractory to treatment with a disease characterized by DUX4 misexpression. For instance, in some embodiments, the subject can be a subject that has failed to respond to treatment with one or more agents for the disease characterized by DUX4 misexpression. In some embodiments, the subject can be a subject that has responded poorly to treatment with one or more agents for the disease characterized by DUX4 misexpression.

### Assay Methods

Compounds can be assayed for activity against the disease characterized by DUX4 misexpression according to any assay known to those of skill in the art.

### Second Therapeutic Agents

In certain embodiments, the compounds and compositions provided herein are useful in methods of treatment of a liver disorder, that comprises further administration of a second agent effective for the treatment of the disorder in a subject in need thereof. The second agent can be any agent known to those of skill in the art to be effective for the treatment of the disorder, including those currently approved by the FDA.

In certain embodiments, a compound provided herein is administered in combination with one second agent. In further embodiments, a second agent is administered in combination with two second agents. In still further embodiments, a second agent is administered in combination with two or more second agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a subject with a disorder. A first therapy (e.g., a prophylactic or therapeutic agent such as a compound provided herein) can be administered before (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or after (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder.

As used herein, the term “synergistic” includes a combination of a compound provided herein and another therapy (e.g., a prophylactic or therapeutic agent) which has been or is currently being used to prevent, manage or treat a disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The active compounds provided herein can be administered in combination or alternation with another therapeutic agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In certain embodiments, a compound for treatment against a disease characterized by DUX4 misexpression has an EC₅₀ of 1 to 15 μM. In certain embodiments, a compound with an EC₅₀ less than 1 to 5 μM is desirable.

Examples of second agents include losmapimod, vitamin C, vitamin E, zinc gluconate, and selenomethionine.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); h, hr, or hrs (hours); min (minutes); TLC (thin layer chromatography); HPLC (high pressure liquid chromatography); THF (tetrahydrofuran); CDCl₃ (deuterated chloroform); DCM (dichloromethane); DMSO (dimethylsulfoxide); DMSO-d₆ (deuterated dimethylsulfoxide); and EtOAc (ethyl acetate).

For all the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through specific examples and are not indicative of the scope of the disclosure.

### Example 1

### Synthesis of N-(6-(Benzo[d]thiazol-2-yl)pyridin-3-yl)-3-(2,5-dioxopyrrolidin-1-yl) propenamide (AT003) (5a)

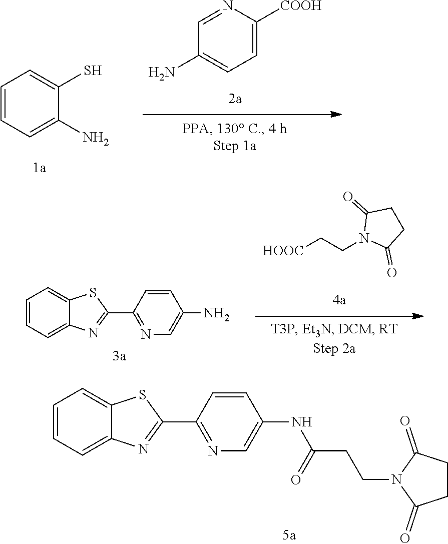 [SMILES: Nc1ccccc1S ; Nc1ccc(C(=O)O)nc1 ; O=C(O)CCN1C(=O)CCC1=O ; Nc1ccc(-c2nc3ccccc3s2)nc1 ; O=C(CCN1C(=O)CCC1=O)Nc1ccc(-c2nc3ccccc3s2)nc1]

### Synthesis of 6-(Benzo[d]thiazol-2-yl)pyridin-3-amine (3)

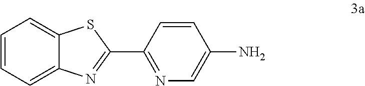 [SMILES: Nc1ccc(-c2nc3ccccc3s2)nc1]

To a mixture of 2-aminobenzenethiol (la) (2 g, 15.98 mmol) and 5-aminopicolinic acid (2) (2.20 g, 15.98 mmol) was added polyphosphoric acid (30 g) at room temperature under a nitrogen atmosphere. The reaction was heated at 130° C. for 4 h. TLC analysis showed the consumption of the starting material. The reaction mixture was quenched with water (50 mL) and slowly adjusted the pH to neutral using saturated sodium hydroxide solution, and the resulting precipitate was filtered and washed with water. The obtained solid was triturated with MTBE and dried under vacuo to afford 6-(benzo[d]thiazol-2-yl)pyridin-3-amine (3a) (1.9 g, 8.36 mmol, 52.3% yield) as a pale yellow solid. LCMS (ESI, +ve mode): 83.94%, Observed: 228.2 (M+H) for C₁₂H₉N₃S, RT: 1.74 min.

### Synthesis of N-(6-(benzo[d]thiazol-2-yl)pyridin-3-yl)-3-(2,5-dioxopyrrolidin-1-yl) propanamide (5a) (AT003)

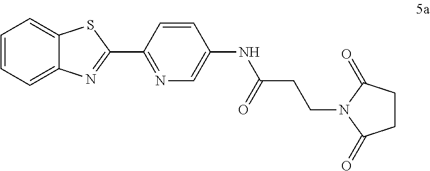 [SMILES: O=C(CCN1C(=O)CCC1=O)Nc1ccc(-c2nc3ccccc3s2)nc1]

To a stirred solution of 6-(benzo[d]thiazol-2-yl)pyridin-3-amine (3a) (100 mg, 0.440 mmol) in dichloromethane (10 mL) was added 3-(2,5-dioxopyrrolidin-1-yl) propanoic acid (4a) (75 mg, 0.440 mmol) under nitrogen atmosphere. The reaction mixture was cooled to 0° C., then triethylamine (0.307 mL, 2.200 mmol) and T3P in EtOAc (0.786 mL, 1.320 mmol, 50% solution) were added. The reaction mixture was stirred at room temperature for 16 h. TLC analysis showed the consumption of the starting material. The reaction mixture was diluted with DCM (15 mL), washed with 10% sodium bicarbonate solution (10 mL) and brine solution (10 mL). The organic layer was dried over anhydrous sodium sulphate and concentrated under reduced pressure to get the crude residue. The obtained residue was purified by preparatory HPLC (0.1% HCOOH in ACN) and lyophilized to afford N-(6-(benzo[d]thiazol-2-yl)pyridin-3-yl)-3-(2,5-dioxopyrrolidin-1-yl) propanamide (38 mg, 0.099 mmol, 22.50% yield) (5a) as a white solid. ¹H-NMR (400 MHZ, DMSO-d₆): δ 10.57 (s, 1H), 8.81 (s, 1H), 8.29 (s, 2H), 8.15 (d, J=8.00 Hz, 1H), 8.07 (d, J=8.00 Hz, 1H), 7.57-7.53 (m, 1H), 7.48 (t, J=7.20 Hz, 1H), 3.71 (t, J=7.20 Hz, 2H), 2.67-2.64 (m, 6H). LCMS (ESI, +ve mode): 99.61%, Observed: 381.1 (M+H) for C₂₁H₁₉N₃O₃S, RT: 2.07 min. HPLC: 99.09%, RT: 3.55 min.

### Example 2

### Synthesis of 4-Acetamido-N-(4-(benzo[b]thiophen-2-yl)-phenyl)-N-methylbutanamide (AT037) (5a)

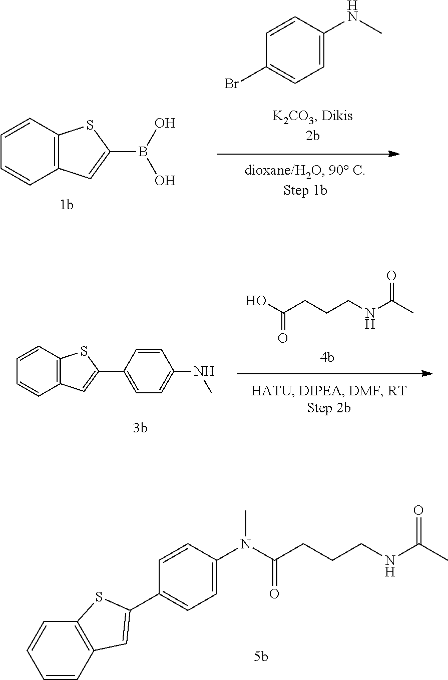

### Synthesis of 4-(Benzo[b]thiophen-2-yl)-N-methylaniline (3b)

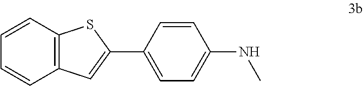 [SMILES: CNc1ccc(-c2cc3ccccc3s2)cc1]

In a 25 mL sealed tube containing a solution of benzo[b]thiophen-2-ylboronic acid (1b) (500 mg, 2.81 mmol) in a mixture of dioxane (6 mL) and water (2 mL) was added 4-bromo-N-methylaniline (2b) (523 mg, 2.81 mmol) and potassium carbonate (1165 mg, 8.43 mmol) at room temperature. The reaction mixture was degassed with nitrogen for 5 min, then bis(triphenylphosphine) palladium (II) dichloride (197 mg, 0.281 mmol) was added. The reaction was stirred at 90° C. for 12 h. TLC analysis showed the consumption of the starting material. The reaction mixture was dissolved in ethyl acetate (50 mL), washed with water (20 mL) and brine solution (20 mL). The organic layer was dried over anhydrous sodium sulfate and concentrated under reduced pressure to get the crude residue. The residue thus obtained was purified by column chromatography on silica gel (230-400 mesh) eluted with 0-15% EtOAc on pet ether to afford 4-(benzo[b]thiophen-2-yl)-N-methylaniline (3b) (300 mg, 1.136 mmol, 40.5% yield) as a white solid. LCMS (ESI, +ve mode): 90.65%, Observed: 240.1 (M+H) for C₁₅H₁₃NS, RT: 3.00 min

### Synthesis of 4-Acetamido-N-(4-(benzo[b]thiophen-2-yl)-phenyl)-N-methylbutanamide (AT037) (5b)

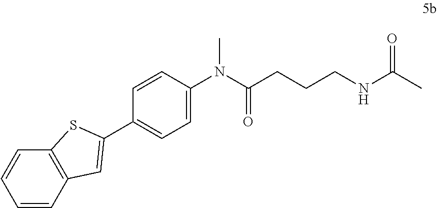 [SMILES: CC(=O)NCCCC(=O)N(C)c1ccc(-c2cc3ccccc3s2)cc1]

To a stirred solution of 4-(benzo[b]thiophen-2-yl)-N-methylaniline (3b) (100 mg, 0.418 mmol) in N,N-dimethylformamide (6 mL) was added 4-acetamidobutanoic acid (4b) (60.7 mg, 0.418 mmol) and DIPEA (0.298 mL, 1.671 mmol) at 0° C. Then HATU (318 mg, 0.836 mmol) was added and stirred for 12 h at room temperature. LCMS analysis showed the consumption of the starting material. The reaction mixture was dissolved in DCM (15 mL), washed with 10% sodium bicarbonate solution (10 mL) and water (10 mL). The organic layer was dried over anhydrous sodium sulfate and concentrated under reduced pressure to get the crude residue. The residue thus obtained was purified by Prep HPLC (0.1% NH₄HCO₃ in ACN) and lyophilized to afford 4-acetamido-N-(4-(benzo[b]thiophen-2-yl)-phenyl)-N-methylbutanamide (16 mg, 0.041 mmol, 10% yield) (AT037) (5b) as a white solid. ¹H-NMR (400 MHZ, DMSO-d₆): δ 8.00 (d, J=7.20 Hz, 1H), 7.92 (s, 1H), 7.88-7.84 (m, 3H), 7.73 (s, 1H), 7.42-7.36 (m, 4H), 3.20 (s, 3H), 2.96-2.94 (m, 2H), 2.11 (s, 2H), 1.73 (s, 3H), 1.61 (t, J=7.20 Hz, 2H). LCMS (ESI, +ve mode): 99.73%, Observed: 367.1 (M+H) for C₂₁H₂₂N₂O₂S, RT: 2.51 min. HPLC: 99.92%, RT: 4.20 min.

### Example 3

### Synthesis of N-(4-(Benzo[d]thiazol-2-yl)phenyl)-4-propionamidobutanamide (AT045) (5c)

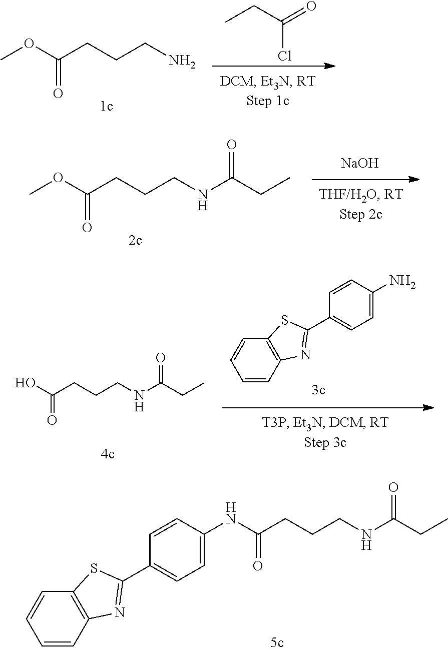

### Synthesis of 4 Methyl 4-Propionamidobutanoate (2c)

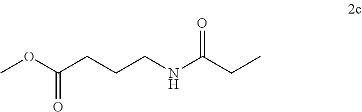

To a solution of methyl 4-aminobutanoate hydrochloride (1c) (500 mg, 3.26 mmol) in DCM (10 mL) were added triethylamine (1.372 mL, 9.77 mmol) and propionyl chloride (301 mg, 3.26 mmol) at 0° C. It was stirred for 16 h at RT. LCMS analysis showed the consumption of the starting material. The reaction mixture was diluted with DCM (15 mL), washed with water (10 mL) and sodium bicarbonate solution (10 mL). The organic layer was dried over anhydrous sodium sulfate and concentrated under reduced pressure to afford methyl 4-propionamidobutanoate (2c) (190 mg, 1.096 mmol, 33.7% yield) as a pale-yellow liquid. It was taken to the next step without further purification. LCMS (ELSD +ve mode): 99.94%, Observed: 174.1 (M+H) for C₈H₁₅NO₃, RT: 1.23 min.

### Synthesis of 4 Methyl 4-Propionamidobutanoate (4c)

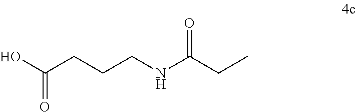

To a stirred solution of methyl 4-propionamidobutanoate (3c) (180 mg, 1.039 mmol) in a mixture of water (4 mL) and THF (4 mL) was added sodium hydroxide (125 mg, 3.12 mmol) at 0° C. It was stirred for 2 h at RT. LCMS analysis showed the completion of the starting material. The reaction mixture was concentrated under reduced pressure to remove THF and acidified (pH 3-4) using 1.5 N HCl and extracted with DCM (2×15 mL). The organic layer was dried over anhydrous sodium sulfate and concentrated under reduced pressure to afford 4-propionamidobutanoic acid (4c) (75 mg, 0.405 mmol, 38.9% yield) as a pale-yellow liquid. It was taken to the next step without further purification. LCMS (ESI, +ve mode): 86%, Observed: 160.3 (M+H) for C₇H₁₃NO₃, RT: 0.94 min.

### Synthesis of N-(4-(Benzo[d]thiazol-2-yl)phenyl)-4-propionamidobutanamide (AT045) (5c)

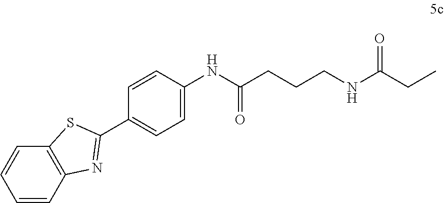 [SMILES: CCC(=O)NCCCC(=O)Nc1ccc(-c2nc3ccccc3s2)cc1]

To a stirred solution of 4-(benzo[b]thiophen-2-yl)-N-methylaniline (3b) (100 mg, 0.418 mmol) in N,N-dimethylformamide (6 mL) was added 4-acetamidobutanoic acid (4b) (60.7 mg, 0.418 mmol) and DIPEA (0.298 mL, 1.671 mmol) at 0° C. Then HATU (318 mg, 0.836 mmol) was added and stirred for 12 h at room temperature. LCMS analysis showed the consumption of the starting material. The reaction mixture was dissolved in DCM (15 mL), washed with 10% sodium bicarbonate solution (10 mL) and water (10 mL). The organic layer was dried over anhydrous sodium sulfate and concentrated under reduced pressure to get the crude residue. The residue thus obtained was purified by Prep HPLC (0.1% NH₄HCO₃ in ACN) and lyophilized to afford 4-acetamido-N-(4-(benzo[b]thiophen-2-yl)-phenyl)-N-methylbutanamide (16 mg, 0.041 mmol, 10% yield) (AT037) (5b) as a white solid. ¹H-NMR (400 MHZ, DMSO-d₆): δ 8.00 (d, J=7.20 Hz, 1H), 7.92 (s, 1H), 7.88-7.84 (m, 3H), 7.73 (s, 1H), 7.42-7.36 (m, 4H), 3.20 (s, 3H), 2.96-2.94 (m, 2H), 2.11 (s, 2H), 1.73 (s, 3H), 1.61 (t, J=7.20 Hz, 2H). LCMS (ESI, +ve mode): 99.73%, Observed: 367.1 (M+H) for C₂₁H₂₂N₂O₂S, RT: 2.51 min. HPLC: 99.92%, RT: 4.20 min.

To a stirred solution of 4-propionamidobutanoic acid (4c) (70 mg, 0.440 mmol) in DCM (5 mL) was added 4-(benzo[d]thiazol-2-yl)-aniline (3c) (100 mg, 0.440 mmol) and triethylamine (0.062 ml, 0.440 mmol) at 0° C. Then T₃P in EtOAc (0.259 ml, 0.440 mmol, 50%) was added and stirred for 12 h at room temperature. TLC analysis showed the consumption of the starting material. The reaction mixture was diluted with DCM (20 mL), washed with 10% sodium bicarbonate solution (10 mL) and brine solution (10 mL). The organic layer was dried over anhydrous sodium sulphate and concentrated under reduced pressure to get the crude residue. The obtained residue was purified by Prep HPLC (0.1% NH₄HCO₃ in ACN) and lyophilized to afford N-(4-(benzo[d]thiazol-2-yl)phenyl)-4-propionamidobutanamide (63 mg, 0.171 mmol, 39.0% yield) (AT045) (5c) as a white solid. ¹H-NMR (400 MHZ, DMSO-d₆): δ 10.25 (s, 1H), 8.13 (d, J=7.20 Hz, 1H), 8.06-8.02 (m, 3H), 7.82-7.80 (m, 3H), 7.56-7.51 (m, 1H), 7.46-7.42 (m, 1H), 3.10 (t, J=6.80 Hz, 2H), 2.37 (t, J=7.60 Hz, 2H), 2.07 (q, J=7.60 Hz, 2H), 1.75-1.72 (m, 2H), 1.00 (t, J=7.60 Hz, 2H). LCMS (ESI, +ve mode): 97.3%, Observed: 368.1 (M+H) for C₂₀H₂₁N₃O₂S, RT: 2.40 min. HPLC: 99.94%, RT: 3.86 min.

### Example 4

### Cell Cytotoxicity 50 Assay Protocol (CC50)

### Preparation

MB200 cells were grown until 80% confluent in F10 media supplemented with rhFGF basic (10 ng/ml), 15% fetal bovine serum, 1% penicillin streptomycin, 1% amphotericin B, and 1 μM dexamethasone. Adherent MB200 cells growing on 10 cm plate were then trypsinized with 1 ml of trypsin. Once the cells detached from the plate, F10 media (10 mL) was added, and the cells were strained through a 70 μm cell strainer.

The cells were counted using a mixture of 10 μL cell suspension with 10 μL of trypan blue. Once the total number of cells were counted, a dilution of 200 k/mL was prepared, and the cells were seeded with 10 k cells/well in a 96-well plate in triplicate, making sure to avoid seeding cells in the edges of the plate to avoid edge effects. Using a multichannel pipet, the 200 k/mL cell dilution (50 μL/well) was added into wells B2-B11, C2-C11, and D2-D11 in triplicate.

Cell Treatment with Inhibitors

Into a deep well block (DWB), the media for the appropriate cell line (450 μL/well) was added into wells 1-10. Into DWB well 2, 10 mM of drug solution (13.5 μL) was added, followed by an additional volume of the medium (225 μL). The well was pipetted up/down four times. A portion (225 μL) was transferred to well 3, and this process was repeated up to well 10. Using a multichannel pipet, a smaller portion (50 μL/well) was removed from all wells in the DWB and then dispensed on top of the cells in each corresponding row. After adding the drug, the plate was incubated at 37° C. for 72 h.

### Reading Luminescence

Using a multi-channel pipet, pre-warmed cell titer glo reagent (Promega) (100 μL) was added into every well. After 5 minutes, the total luminescence was measured with a luminometer. CC50 was determined with GraphPad prism template.

### Example 5

### EC₅₀ Protocol

### Preparation of Transfection Complex

This example provides a representative protocol for a single well of a 96-well plate. It can be scaled up as necessary for treatment of multiple wells.

For negative controls, a well containing reporter DNA, Renilla DNA, and turbofect (without expression vector) were used. For positive controls, wells containing cells that were not treated with any inhibitor were used. They were transfected with the complete DNA lipid complex as set forth below.

The DNA-lipid complex for one well of the 96-well plate is prepared by adding to a tube 100 ng of the transfection factor (TF) expression vector, 100 ng of TF reporter DNA, 10 ng of Renilla reporter DNA, 0.4 mL Turbofect transfection reagent, and 25 mL of the medium without any serum or penicillin/streptomycin. The combination was mixed by gentle flicking of the tube, and the mixture was pipetted into a well of a 96-well plate. The plate was then gently tapped to spread the combination evenly on the bottom of the well. The well was incubated for 30 min.

### Cell Preparation

HEK293 cells were grown until 80% confluent in DMEM supplemented with 10% fetal bovine serum, 1% penicillin streptomycin, and 1% amphotericin B. The adherent cells growing on a 10 cm plate were trypsinized with 1 mL of prewarmed trypsin. Once the cells detached from the plate, 10 mL of medium was added, and the cells were strained through a 70 mm cell strainer.

The cells were counted using a mixture of 10 mL cell suspension with 10 mL of trypan blue. Once the total number of cells are counted, a dilution of 150 k/mL was prepared.

### Transfection

After incubation of the transfection complex was complete, 155 mL of HEK293s (23.25 k total cells) were gently dispensed on top of the wells containing the 25 mL transfection complex, producing a total volume of about 180 mL. The mixture was then incubated for 24 h.

Cell Treatment with Inhibitors

To treat cells with each inhibitor, the inhibitors were prepared in the same growth medium. In a deep well block, the regular medium with serum was added to well 1 (135 mL) and to wells 2 through 10 (100 mL). To the first well was added 15 mL of 10 nM inhibitor. The final concentration of the drug in well 1 was 1 nM. The wells were pipeted up and down, and 50 mL was transferred from well 1 to well 2 (1/3 dilution). The well was mixed, and the seriation dilution was repeated with the remaining wells. Once the serial dilution was complete, using a multichannel pipet, 20 mL of each inhibitor were transferred to designated wells in a 96-well plate. After the inhibitor was added, the cells were incubated at 37° C. for 24 h.

### Measuring Relative Luciferase Activity Using a Luciferase Assay System

Before the assay, a sufficient amount of 1× passive lysis buffer was prepared. The luciferase substrate buffer (Promega) was thawed completely and then mixed with the lyophilized luciferase substrate. The stop buffer (Promega) was also completely thawed.

(1) Cell lysis: After treatment of the cells was complete, all the cell media is removed by flipping the 96-well plate over and tapping it dry on a dry paper towel. Immediately afterwards, the 1× passive lysis buffer (25 μL) was added. The plates were then placed on a rocker for 15 minutes at medium speed.

(2)Measuring luciferase signal: The luciferase substrate solution for all wells was placed in a solution basin. The luciferase substrate solution was then added to every well (100 μL/well) using a multichannel pipette. The total luminescence was then read immediately using a plate reader.

(3)Measuring Renilla signal: The “complete stop solution” was prepared in a solution basin by mixing the buffer with the 50× stop solution substrate (Promega) to make the final 1× solution. The complete stop solution was added to each well (50 μL/well) using a multichannel pipette. The total luminescence was then read immediately using a plate reader.

### Measuring Relative Luciferase Units

Using a spreadsheet (e.g., Excel), the luciferase signal was divided by the renilla signal to get a relative luciferase unit.

### Example 6

### Synthesis and Activity of Exemplary Compounds

Table 6-1 below shows additional exemplary compounds prepared according to the methods of the preceding examples as well as the compounds' activities.

| | | | | | qPCR |
|---|---|---|---|---|---|
| | | 6 hr | CC50 | CC50 | MBD3L2 |
| | | DUX4 | MB200 | HEK | Inhib at |
| Structure | ID | EC₅₀ | (mM) | (mM) | 11 mM |
| 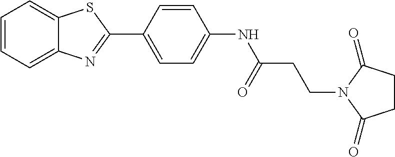 [SMILES: O=C(CCN1C(=O)CCC1=O)Nc1ccc(-c2nc3ccccc3s2)cc1] | 1 | B | B | A | YES |
| 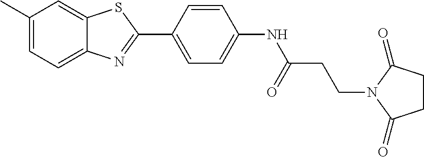 [SMILES: Cc1ccc2nc(-c3ccc(NC(=O)CCN4C(=O)CCC4=O)cc3)sc2c1] | 2 | B | B | A | NO |
| 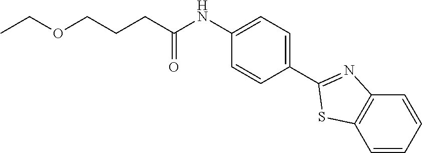 [SMILES: CCOCCCC(=O)Nc1ccc(-c2nc3ccccc3s2)cc1] | 3 | B | B | B | NO |
| 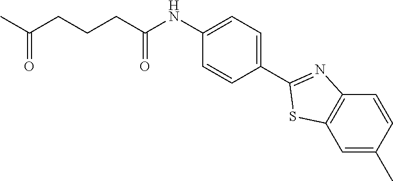 [SMILES: CC(=O)CCCC(=O)Nc1ccc(-c2nc3ccc(C)cc3s2)cc1] | 4 | C | A | A | NO |
| 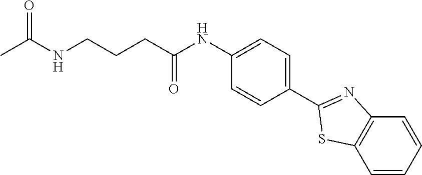 [SMILES: CC(=O)NCCCC(=O)Nc1ccc(-c2nc3ccccc3s2)cc1] | 5 | C | A | A | YES |
| 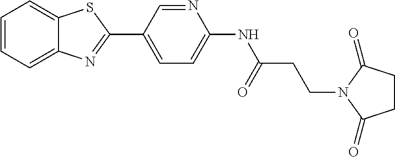 [SMILES: O=C(CCN1C(=O)CCC1=O)Nc1ccc(-c2nc3ccccc3s2)cn1] | 6 | B | A | A | NO |
| 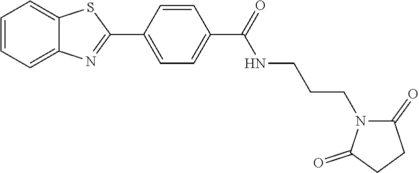 [SMILES: O=C(NCCCN1C(=O)CCC1=O)c1ccc(-c2nc3ccccc3s2)cc1] | 7 | A | A | A | YES |
| 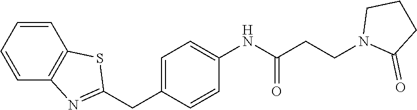 [SMILES: O=C(CCN1CCCC1=O)Nc1ccc(Cc2nc3ccccc3s2)cc1] | 8 | A | A | A | YES |
| 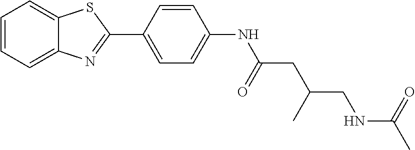 [SMILES: CC(=O)NCC(C)CC(=O)Nc1ccc(-c2nc3ccccc3s2)cc1] | 9 | C | A | A | YES |
| 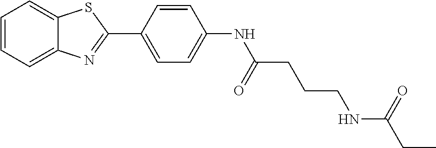 [SMILES: CCC(=O)NCCCC(=O)Nc1ccc(-c2nc3ccccc3s2)cc1] | 10 | C | A | A | YES |
| 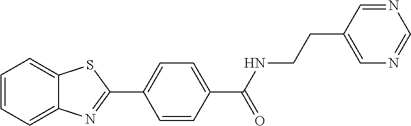 [SMILES: O=C(NCCc1cncnc1)c1ccc(-c2nc3ccccc3s2)cc1] | 11 | A | A | A | YES |
| 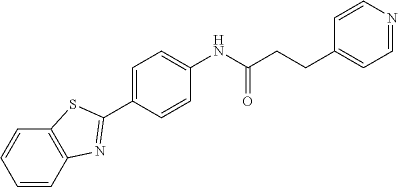 [SMILES: O=C(CCc1ccncc1)Nc1ccc(-c2nc3ccccc3s2)cc1] | 12 | C | A | A | NO |
| 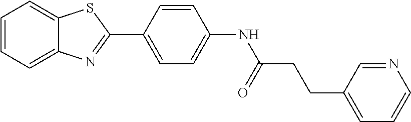 [SMILES: O=C(CCc1cccnc1)Nc1ccc(-c2nc3ccccc3s2)cc1] | 13 | C | A | A | YES |
| 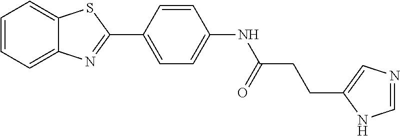 [SMILES: O=C(CCc1cnc[nH]1)Nc1ccc(-c2nc3ccccc3s2)cc1] | 14 | C | A | A | YES |
| 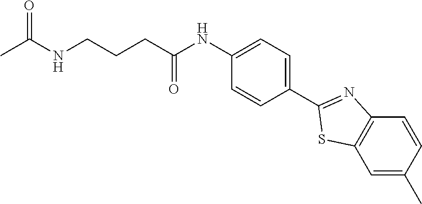 [SMILES: CC(=O)NCCCC(=O)Nc1ccc(-c2nc3ccc(C)cc3s2)cc1] | 15 | C | A | A | YES |
| 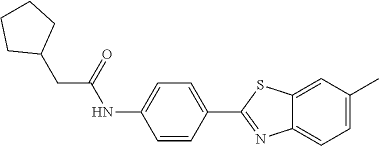 [SMILES: Cc1ccc2nc(-c3ccc(NC(=O)CC4CCCC4)cc3)sc2c1] | 16 | B | A | A | YES |
| 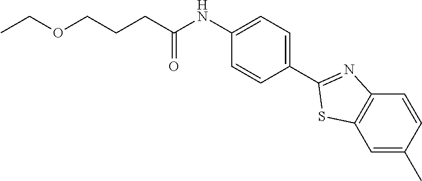 [SMILES: CCOCCCC(=O)Nc1ccc(-c2nc3ccc(C)cc3s2)cc1] | 17 | C | B | B | NO |
| 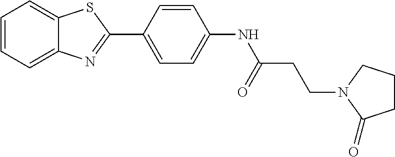 [SMILES: O=C(CCN1CCCC1=O)Nc1ccc(-c2nc3ccccc3s2)cc1] | 18 | B | A | A | YES |
| 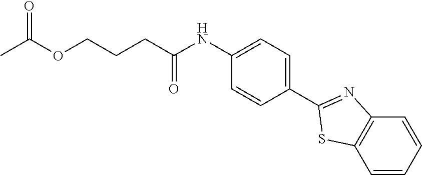 [SMILES: CC(=O)OCCCC(=O)Nc1ccc(-c2nc3ccccc3s2)cc1] | 19 | C | A | A | NO |
| 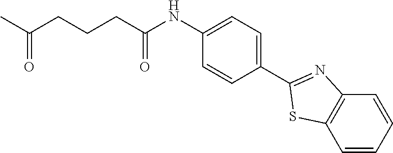 [SMILES: CC(=O)CCCC(=O)Nc1ccc(-c2nc3ccccc3s2)cc1] | 20 | C | A | A | NO |
| 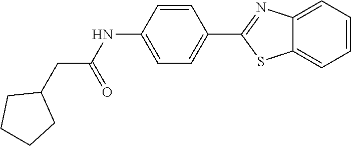 [SMILES: O=C(CC1CCCC1)Nc1ccc(-c2nc3ccccc3s2)cc1] | 21 | C | A | A | YES |
| 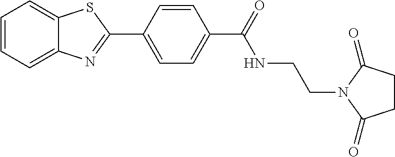 [SMILES: O=C(NCCN1C(=O)CCC1=O)c1ccc(-c2nc3ccccc3s2)cc1] | 22 | A | A | A | YES |
| 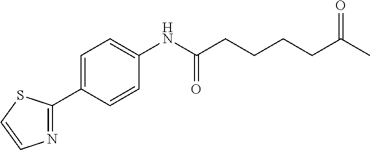 [SMILES: CC(=O)CCCCC(=O)Nc1ccc(-c2nccs2)cc1] | 23 | A | A | A | YES |
| 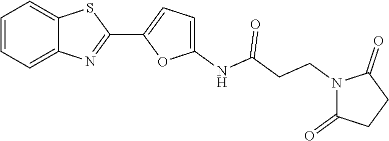 [SMILES: O=C(CCN1C(=O)CCC1=O)Nc1ccc(-c2nc3ccccc3s2)o1] | 24 | B | A | A | NO |
| 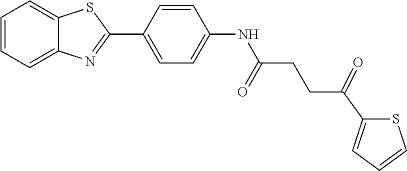 [SMILES: O=C(CCC(=O)c1cccs1)Nc1ccc(-c2nc3ccccc3s2)cc1] | 25 | A | A | A | NO |
| 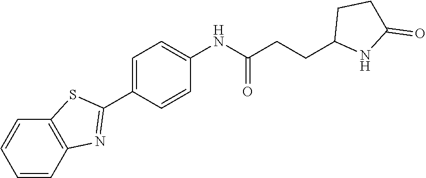 [SMILES: O=C(CCC1CCC(=O)N1)Nc1ccc(-c2nc3ccccc3s2)cc1] | 26 | C | B | A | YES |
| 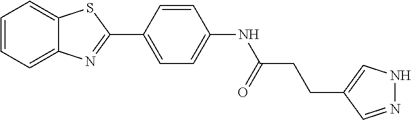 [SMILES: O=C(CCc1cn[nH]c1)Nc1ccc(-c2nc3ccccc3s2)cc1] | 27 | C | A | A | YES |
| 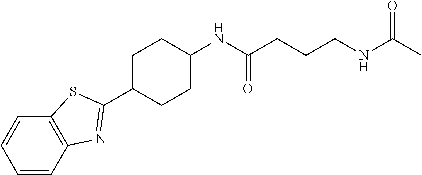 [SMILES: CC(=O)NCCCC(=O)NC1CCC(c2nc3ccccc3s2)CC1] | 28 | A | A | A | YES |
| 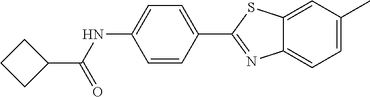 [SMILES: Cc1ccc2nc(-c3ccc(NC(=O)C4CCC4)cc3)sc2c1] | 29 | C | B | A | YES |
| 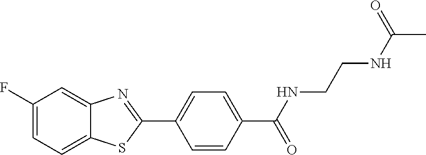 [SMILES: CC(=O)NCCNC(=O)c1ccc(-c2nc3cc(F)ccc3s2)cc1] | 30 | B | A | A | YES |
| 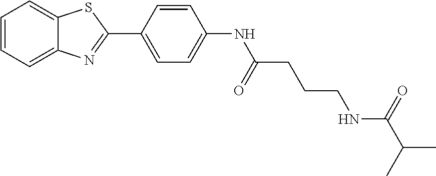 [SMILES: CC(C)C(=O)NCCCC(=O)Nc1ccc(-c2nc3ccccc3s2)cc1] | 31 | C | A | A | YES |
| 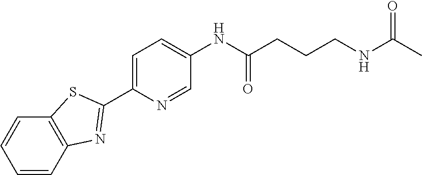 [SMILES: CC(=O)NCCCC(=O)Nc1ccc(-c2nc3ccccc3s2)nc1] | 32 | C | A | A | YES |
| 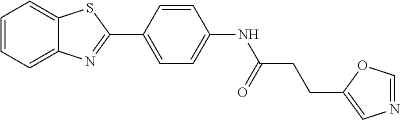 [SMILES: O=C(CCc1cnco1)Nc1ccc(-c2nc3ccccc3s2)cc1] | 33 | C | A | A | YES |
| 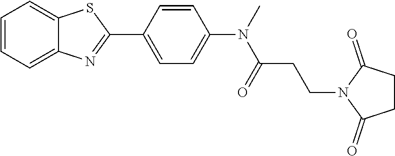 [SMILES: CN(C(=O)CCN1C(=O)CCC1=O)c1ccc(-c2nc3ccccc3s2)cc1] | 34 | B | A | A | YES |
| 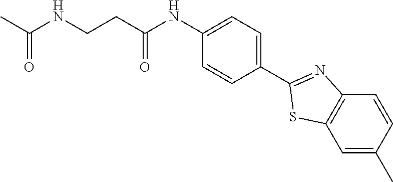 [SMILES: CC(=O)NCCC(=O)Nc1ccc(-c2nc3ccc(C)cc3s2)cc1] | 35 | C | A | A | NO |
| 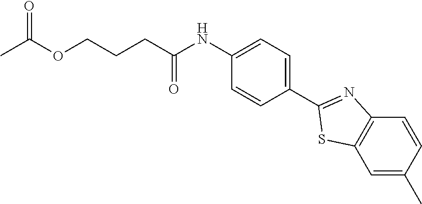 [SMILES: CC(=O)OCCCC(=O)Nc1ccc(-c2nc3ccc(C)cc3s2)cc1] | 36 | C | A | B | YES |
| 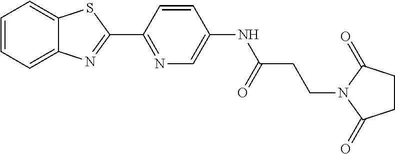 [SMILES: O=C(CCN1C(=O)CCC1=O)Nc1ccc(-c2nc3ccccc3s2)nc1] | 37 | B | A | A | YES |
| 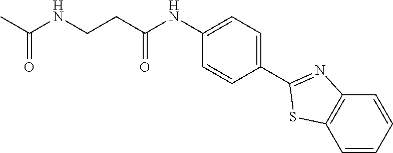 [SMILES: CC(=O)NCCC(=O)Nc1ccc(-c2nc3ccccc3s2)cc1] | 38 | C | A | A | YES |
| 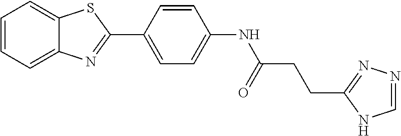 [SMILES: O=C(CCc1nnc[nH]1)Nc1ccc(-c2nc3ccccc3s2)cc1] | 39 | C | A | A | YES |
| 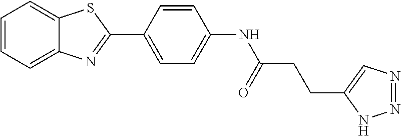 [SMILES: O=C(CCc1cnn[nH]1)Nc1ccc(-c2nc3ccccc3s2)cc1] | 40 | C | A | A | YES |
| 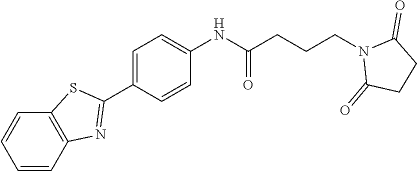 [SMILES: O=C(CCCN1C(=O)CCC1=O)Nc1ccc(-c2nc3ccccc3s2)cc1] | 41 | C | A | A | YES |
| 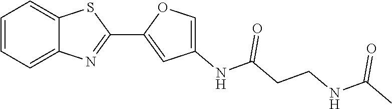 [SMILES: CC(=O)NCCC(=O)Nc1coc(-c2nc3ccccc3s2)c1] | 42 | B | A | A | NO |
| 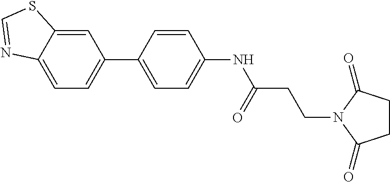 [SMILES: O=C(CCN1C(=O)CCC1=O)Nc1ccc(-c2ccc3ncsc3c2)cc1] | 43 | A | A | A | NO |
| 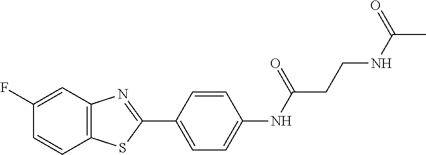 [SMILES: CC(=O)NCCC(=O)Nc1ccc(-c2nc3cc(F)ccc3s2)cc1] | 44 | C | A | A | YES |
| 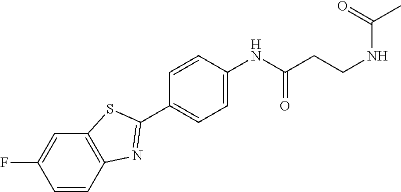 [SMILES: CC(=O)NCCC(=O)Nc1ccc(-c2nc3ccc(F)cc3s2)cc1] | 45 | C | A | A | NO |
| 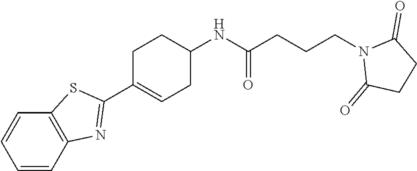 [SMILES: O=C(CCCN1C(=O)CCC1=O)NC1CC=C(c2nc3ccccc3s2)CC1] | 46 | C | A | A | YES |
| 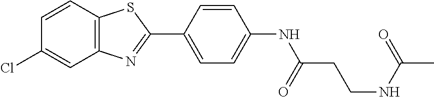 [SMILES: CC(=O)NCCC(=O)Nc1ccc(-c2nc3cc(Cl)ccc3s2)cc1] | 47 | B | A | A | NO |
| 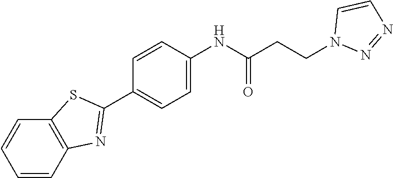 [SMILES: O=C(CCn1ccnn1)Nc1ccc(-c2nc3ccccc3s2)cc1] | 48 | C | A | A | NO |
| 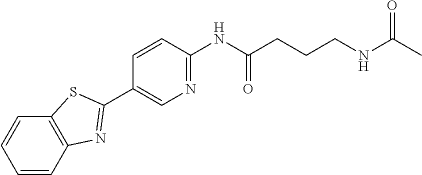 [SMILES: CC(=O)NCCCC(=O)Nc1ccc(-c2nc3ccccc3s2)cn1] | 49 | C | A | A | YES |
| 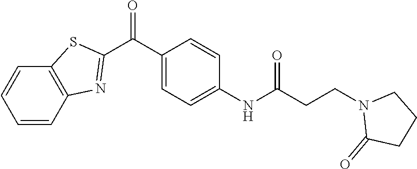 [SMILES: O=C(CCN1CCCC1=O)Nc1ccc(C(=O)c2nc3ccccc3s2)cc1] | 50 | B | A | A | NO |
| 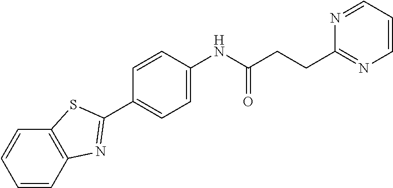 [SMILES: O=C(CCc1ncccn1)Nc1ccc(-c2nc3ccccc3s2)cc1] | 51 | C | A | A | NO |
| 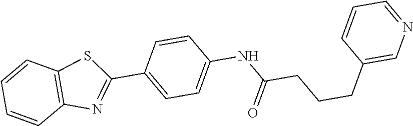 [SMILES: O=C(CCCc1cccnc1)Nc1ccc(-c2nc3ccccc3s2)cc1] | 52 | C | A | A | YES |
| 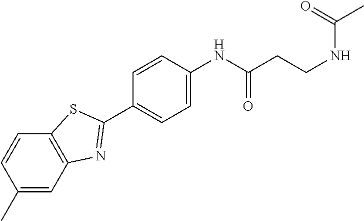 [SMILES: CC(=O)NCCC(=O)Nc1ccc(-c2nc3cc(C)ccc3s2)cc1] | 53 | C | A | A | YES |
| 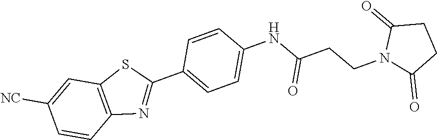 [SMILES: N#Cc1ccc2nc(-c3ccc(NC(=O)CCN4C(=O)CCC4=O)cc3)sc2c1] | 54 | B | B | A | YES |
| 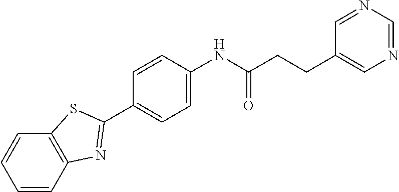 [SMILES: O=C(CCc1cncnc1)Nc1ccc(-c2nc3ccccc3s2)cc1] | 55 | C | A | A | YES |
| 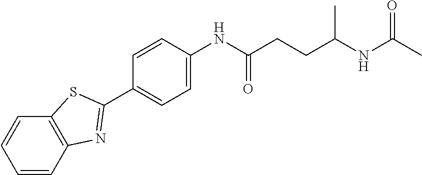 [SMILES: CC(=O)NC(C)CCC(=O)Nc1ccc(-c2nc3ccccc3s2)cc1] | 56 | C | A | A | YES |
| 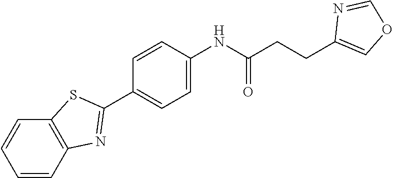 [SMILES: O=C(CCc1cocn1)Nc1ccc(-c2nc3ccccc3s2)cc1] | 57 | C | A | A | NO |
| 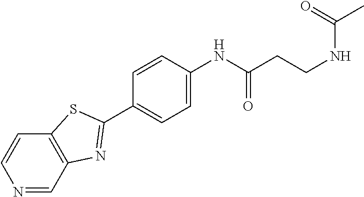 [SMILES: CC(=O)NCCC(=O)Nc1ccc(-c2nc3cnccc3s2)cc1] | 58 | C | A | A | NO |
| 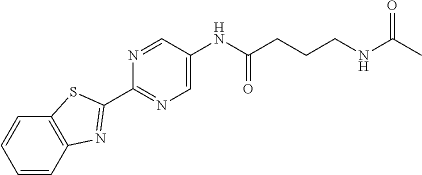 [SMILES: CC(=O)NCCCC(=O)Nc1cnc(-c2nc3ccccc3s2)nc1] | 59 | B | A | A | YES |
| 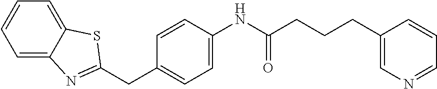 [SMILES: O=C(CCCc1cccnc1)Nc1ccc(Cc2nc3ccccc3s2)cc1] | 60 | C | A | A | YES |
| 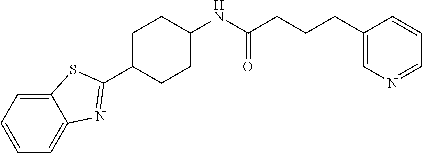 [SMILES: O=C(CCCc1cccnc1)NC1CCC(c2nc3ccccc3s2)CC1] | 61 | C | A | A | YES |
| 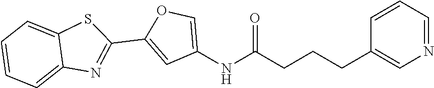 [SMILES: O=C(CCCc1cccnc1)Nc1coc(-c2nc3ccccc3s2)c1] | 62 | C | A | A | YES |
| 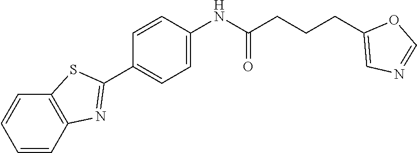 [SMILES: O=C(CCCc1cnco1)Nc1ccc(-c2nc3ccccc3s2)cc1] | 63 | C | A | B | NO |
| 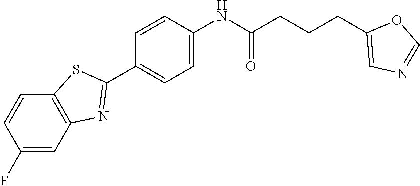 [SMILES: O=C(CCCc1cnco1)Nc1ccc(-c2nc3cc(F)ccc3s2)cc1] | 64 | C | A | B | YES |
| 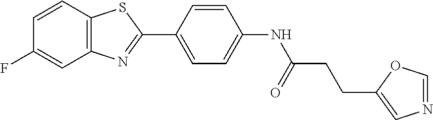 [SMILES: O=C(CCc1cnco1)Nc1ccc(-c2nc3cc(F)ccc3s2)cc1] | 65 | C | A | A | YES |
| 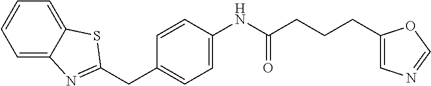 [SMILES: O=C(CCCc1cnco1)Nc1ccc(Cc2nc3ccccc3s2)cc1] | 66 | A | A | A | YES |
| 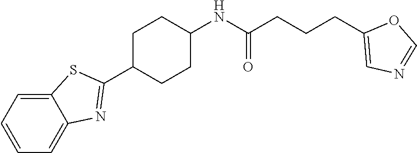 [SMILES: O=C(CCCc1cnco1)NC1CCC(c2nc3ccccc3s2)CC1] | 67 | B | A | A | YES |
| 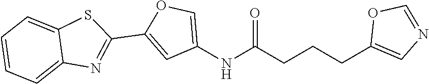 [SMILES: O=C(CCCc1cnco1)Nc1coc(-c2nc3ccccc3s2)c1] | 68 | C | A | A | NO |
| 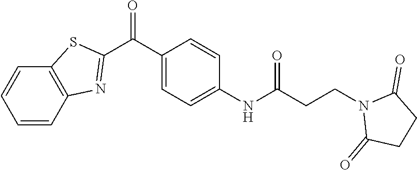 [SMILES: O=C(CCN1C(=O)CCC1=O)Nc1ccc(C(=O)c2nc3ccccc3s2)cc1] | 69 | A | B | A | NO |
| 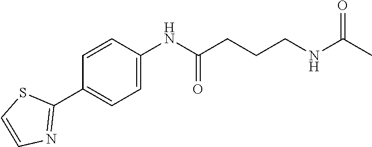 [SMILES: CC(=O)NCCCC(=O)Nc1ccc(-c2nccs2)cc1] | 70 | A | A | A | YES |
| 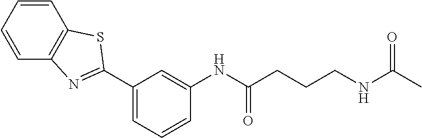 [SMILES: CC(=O)NCCCC(=O)Nc1cccc(-c2nc3ccccc3s2)c1] | 71 | A | A | A | YES |
| 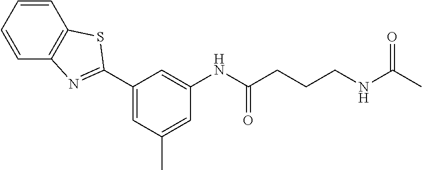 [SMILES: CC(=O)NCCCC(=O)Nc1cc(C)cc(-c2nc3ccccc3s2)c1] | 72 | A | A | A | YES |
| 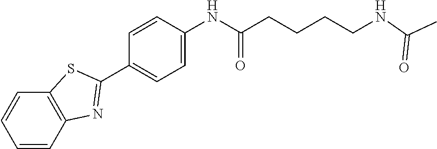 [SMILES: CC(=O)NCCCCC(=O)Nc1ccc(-c2nc3ccccc3s2)cc1] | 73 | C | A | A | YES |
| 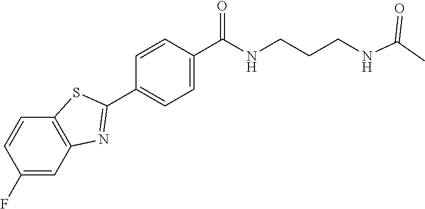 [SMILES: CC(=O)NCCCNC(=O)c1ccc(-c2nc3cc(F)ccc3s2)cc1] | 74 | C | A | A | YES |
| 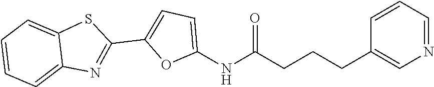 [SMILES: O=C(CCCc1cccnc1)Nc1ccc(-c2nc3ccccc3s2)o1] | 75 | C | A | A | YES |
| 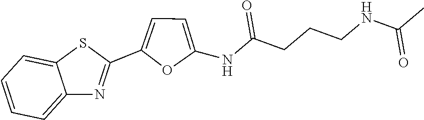 [SMILES: CC(=O)NCCCC(=O)Nc1ccc(-c2nc3ccccc3s2)o1] | 76 | B | A | A | YES |
| 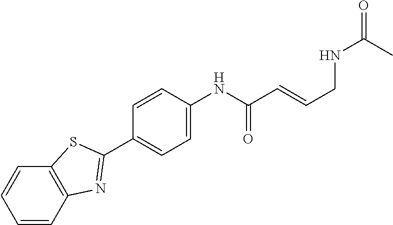 [SMILES: CC(=O)NC/C=C/C(=O)Nc1ccc(-c2nc3ccccc3s2)cc1] | 77 | C \| | A | A | YES |
| 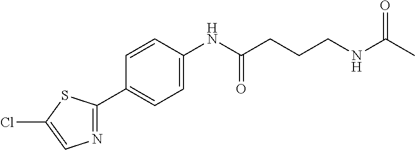 [SMILES: CC(=O)NCCCC(=O)Nc1ccc(-c2ncc(Cl)s2)cc1] | 78 | B | A | A | NO |
| 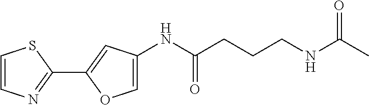 [SMILES: CC(=O)NCCCC(=O)Nc1coc(-c2nccs2)c1] | 79 | A | A | A | NO |
| 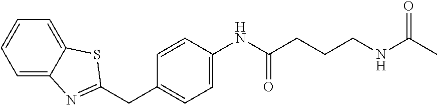 [SMILES: CC(=O)NCCCC(=O)Nc1ccc(Cc2nc3ccccc3s2)cc1] | 80 | A | A | A | NO |
| 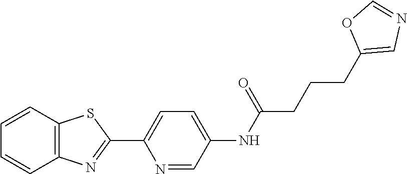 [SMILES: O=C(CCCc1cnco1)Nc1ccc(-c2nc3ccccc3s2)nc1] | 81 | C | A | A | YES |
| 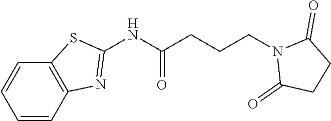 [SMILES: O=C(CCCN1C(=O)CCC1=O)Nc1nc2ccccc2s1] | 82 | C | A | A | NO |
| 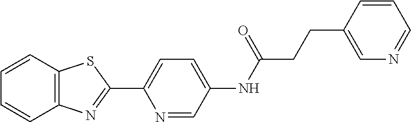 [SMILES: O=C(CCc1cccnc1)Nc1ccc(-c2nc3ccccc3s2)nc1] | 83 | C | A | A | YES |
| 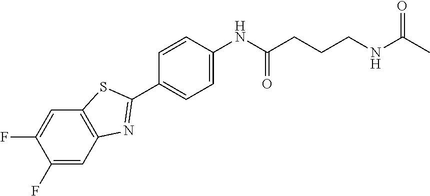 [SMILES: CC(=O)NCCCC(=O)Nc1ccc(-c2nc3cc(F)c(F)cc3s2)cc1] | 84 | C | A | A | YES |
| 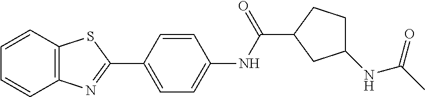 [SMILES: CC(=O)NC1CCC(C(=O)Nc2ccc(-c3nc4ccccc4s3)cc2)C1] | 85 | C | A | A | YES |
| 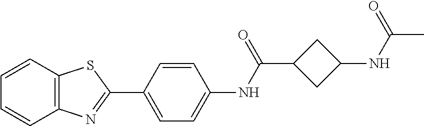 [SMILES: CC(=O)NC1CC(C(=O)Nc2ccc(-c3nc4ccccc4s3)cc2)C1] | 86 | C | A | A | YES |
| 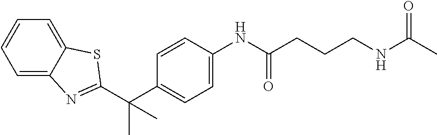 [SMILES: CC(=O)NCCCC(=O)Nc1ccc(C(C)(C)c2nc3ccccc3s2)cc1] | 87 | B | A | A | YES |
| 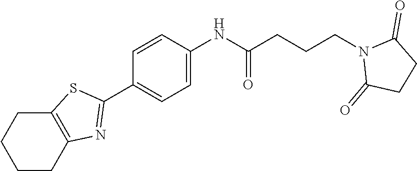 [SMILES: O=C(CCCN1C(=O)CCC1=O)Nc1ccc(-c2nc3c(s2)CCCC3)cc1] | 88 | C | A | A | YES |
| 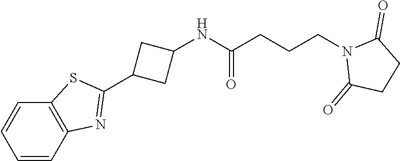 [SMILES: O=C(CCCN1C(=O)CCC1=O)NC1CC(c2nc3ccccc3s2)C1] | 89 | A | A | A | YES |
| 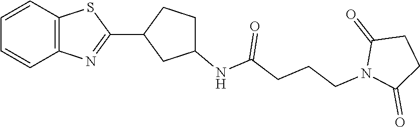 [SMILES: O=C(CCCN1C(=O)CCC1=O)NC1CCC(c2nc3ccccc3s2)C1] | 90 | A | A | A | YES |
| 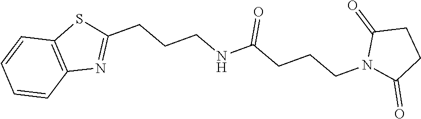 [SMILES: O=C(CCCN1C(=O)CCC1=O)NCCCc1nc2ccccc2s1] | 91 | A | A | A | YES |
| 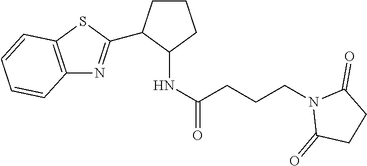 [SMILES: O=C(CCCN1C(=O)CCC1=O)NC1CCCC1c1nc2ccccc2s1] | 92 | A | A | A | YES |
| 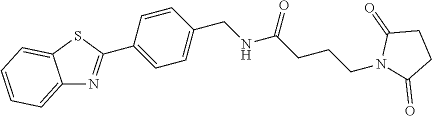 [SMILES: O=C(CCCN1C(=O)CCC1=O)NCc1ccc(-c2nc3ccccc3s2)cc1] | 93 | B | A | A | YES |
| 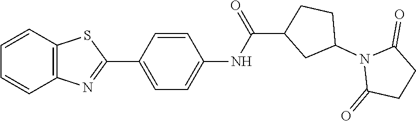 [SMILES: O=C(Nc1ccc(-c2nc3ccccc3s2)cc1)C1CCC(N2C(=O)CCC2=O)C1] | 94 | C | A | A | YES |
| 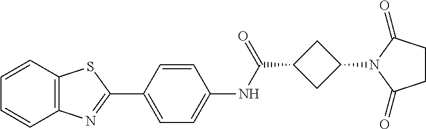 [SMILES: O=C1CCC(=O)N1[C@H]1C[C@@H](C(=O)Nc2ccc(-c3nc4ccccc4s3)cc2)C1] | 95 | A | A | A | NO |
| 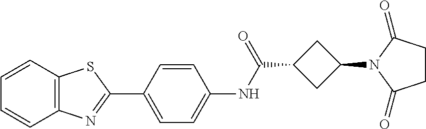 [SMILES: O=C1CCC(=O)N1[C@H]1C[C@H](C(=O)Nc2ccc(-c3nc4ccccc4s3)cc2)C1] | 96 | C | A | A | NO |
| 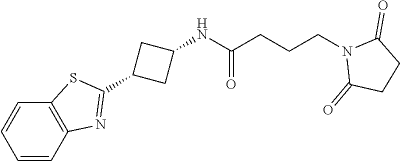 [SMILES: O=C(CCCN1C(=O)CCC1=O)N[C@H]1C[C@@H](c2nc3ccccc3s2)C1] | 97 | A | A | A | YES |
| 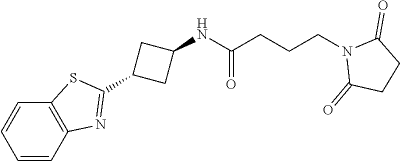 [SMILES: O=C(CCCN1C(=O)CCC1=O)N[C@H]1C[C@H](c2nc3ccccc3s2)C1] | 98 | A | A | A | NO |
| 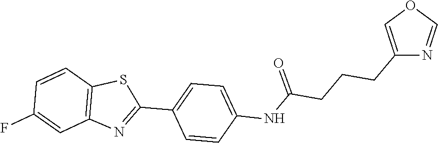 [SMILES: O=C(CCCc1cocn1)Nc1ccc(-c2nc3cc(F)ccc3s2)cc1] | 99 | C | A | A | NO |
| 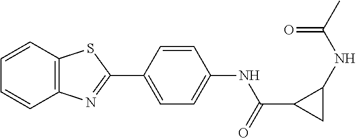 [SMILES: CC(=O)NC1CC1C(=O)Nc1ccc(-c2nc3ccccc3s2)cc1] | 100 | B | A | A | NO |
| 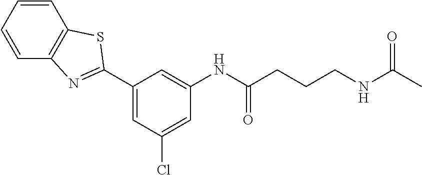 [SMILES: CC(=O)NCCCC(=O)Nc1cc(Cl)cc(-c2nc3ccccc3s2)c1] | 101 | A | A | A | YES |
| 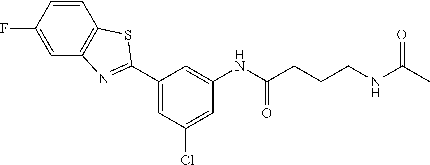 [SMILES: CC(=O)NCCCC(=O)Nc1cc(Cl)cc(-c2nc3cc(F)ccc3s2)c1] | 102 | A | A | A | YES |
| 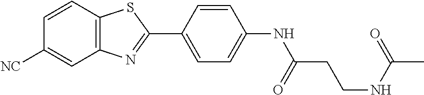 [SMILES: CC(=O)NCCC(=O)Nc1ccc(-c2nc3cc(C#N)ccc3s2)cc1] | 103 | C | A | A | YES |
| 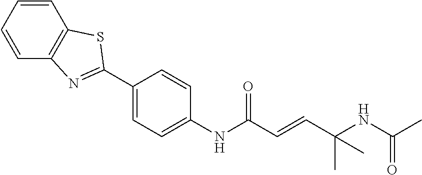 [SMILES: CC(=O)NC(C)(C)/C=C/C(=O)Nc1ccc(-c2nc3ccccc3s2)cc1] | 104 | C | A | A | YES |
| 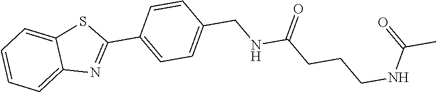 [SMILES: CC(=O)NCCCC(=O)NCc1ccc(-c2nc3ccccc3s2)cc1] | 105 | B | A | A | YES |
| 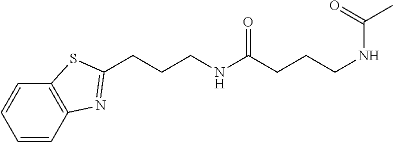 [SMILES: CC(=O)NCCCC(=O)NCCCc1nc2ccccc2s1] | 106 | A | A | A | YES |
| 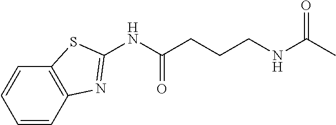 [SMILES: CC(=O)NCCCC(=O)Nc1nc2ccccc2s1] | 107 | A | A | A | YES |
| 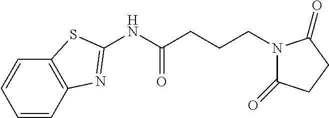 [SMILES: O=C(CCCN1C(=O)CCC1=O)Nc1nc2ccccc2s1] | 108 | A | A | A | YES |
| 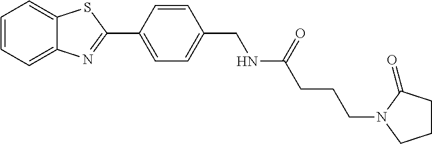 [SMILES: O=C(CCCN1CCCC1=O)NCc1ccc(-c2nc3ccccc3s2)cc1] | 109 | B | A | A | YES |
| 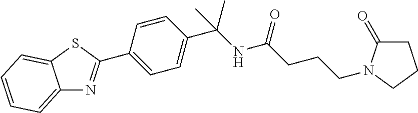 [SMILES: CC(C)(NC(=O)CCCN1CCCC1=O)c1ccc(-c2nc3ccccc3s2)cc1] | 110 | A | A | A | YES |
| 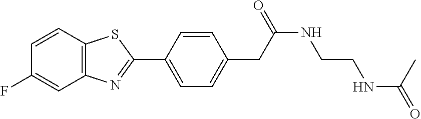 [SMILES: CC(=O)NCCNC(=O)Cc1ccc(-c2nc3cc(F)ccc3s2)cc1] | 111 | B | A | A | YES |
| 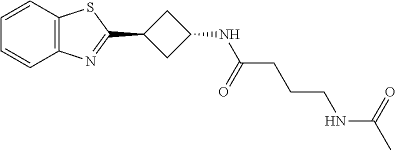 [SMILES: CC(=O)NCCCC(=O)N[C@H]1C[C@H](c2nc3ccccc3s2)C1] | 112 | A | A | A | YES |
| 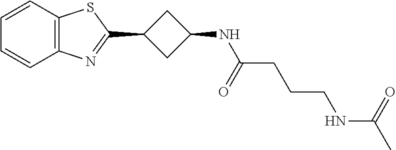 [SMILES: CC(=O)NCCCC(=O)N[C@H]1C[C@@H](c2nc3ccccc3s2)C1] | 113 | A | A | A | YES |
| 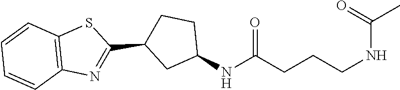 [SMILES: CC(=O)NCCCC(=O)N[C@@H]1CC[C@H](c2nc3ccccc3s2)C1] | 114 | A | A | A | YES |
| 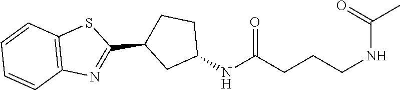 [SMILES: CC(=O)NCCCC(=O)N[C@H]1CC[C@H](c2nc3ccccc3s2)C1] | 115 | A | A | A | YES |
| 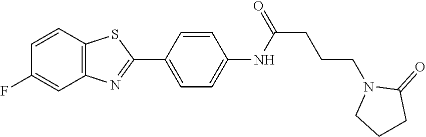 [SMILES: O=C(CCCN1CCCC1=O)Nc1ccc(-c2nc3cc(F)ccc3s2)cc1] | 116 | B | A | A | YES |
| 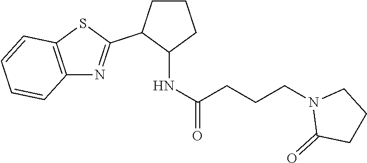 [SMILES: O=C(CCCN1CCCC1=O)NC1CCCC1c1nc2ccccc2s1] | 117 | A | A | A | YES |
| 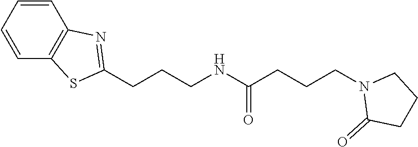 [SMILES: O=C(CCCN1CCCC1=O)NCCCc1nc2ccccc2s1] | 118 | A | A | A | YES |
| 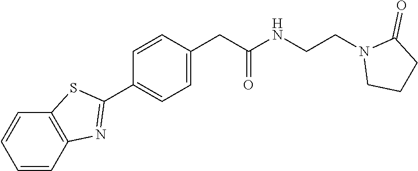 [SMILES: O=C(Cc1ccc(-c2nc3ccccc3s2)cc1)NCCN1CCCC1=O] | 119 | A | A | A | YES |
| 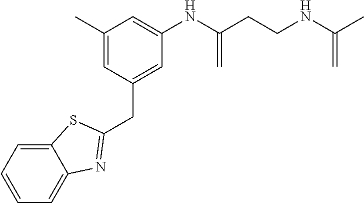 [SMILES: C=C(C)NCCC(=C)Nc1cc(C)cc(Cc2nc3ccccc3s2)c1] | 120 | A | A | A | YES |
| 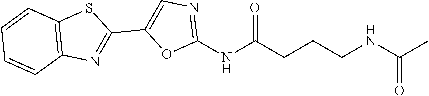 [SMILES: CC(=O)NCCCC(=O)Nc1ncc(-c2nc3ccccc3s2)o1] | 121 | A | A | A | NO |
| 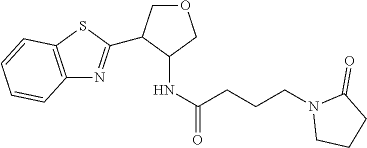 [SMILES: O=C(CCCN1CCCC1=O)NC1COCC1c1nc2ccccc2s1] | 122 | A | A | A | NO |
| 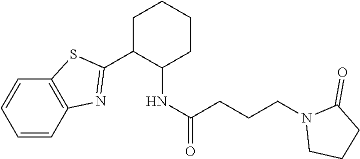 [SMILES: O=C(CCCN1CCCC1=O)NC1CCCCC1c1nc2ccccc2s1] | 123 | A | A | A | NO |
| 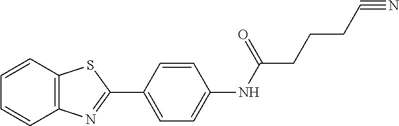 [SMILES: N#CCCCC(=O)Nc1ccc(-c2nc3ccccc3s2)cc1] | 124 | C | A | A | NO |
| 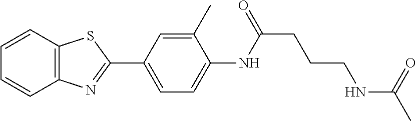 [SMILES: CC(=O)NCCCC(=O)Nc1ccc(-c2nc3ccccc3s2)cc1C] | 125 | C | A | A | NO |
| 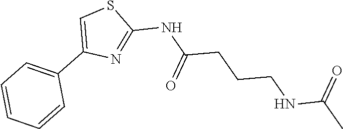 [SMILES: CC(=O)NCCCC(=O)Nc1nc(-c2ccccc2)cs1] | 126 | A | A | A | YES |
| 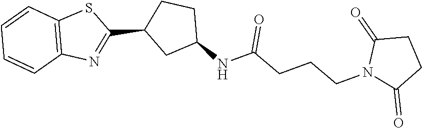 [SMILES: O=C(CCCN1C(=O)CCC1=O)N[C@@H]1CC[C@H](c2nc3ccccc3s2)C1] | 127 | A | A | A | NO |
| 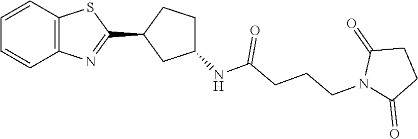 [SMILES: O=C(CCCN1C(=O)CCC1=O)N[C@H]1CC[C@H](c2nc3ccccc3s2)C1] | 128 | A | A | A | NO |
| 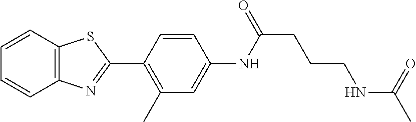 [SMILES: CC(=O)NCCCC(=O)Nc1ccc(-c2nc3ccccc3s2)c(C)c1] | 129 | C | A | A | YES |
| 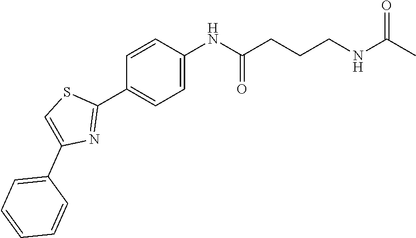 [SMILES: CC(=O)NCCCC(=O)Nc1ccc(-c2nc(-c3ccccc3)cs2)cc1] | 130 | B | A | A | YES |
| 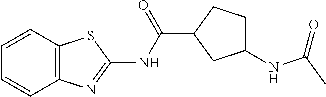 [SMILES: CC(=O)NC1CCC(C(=O)Nc2nc3ccccc3s2)C1] | 131 | A | A | A | YES |
| 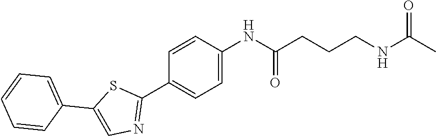 [SMILES: CC(=O)NCCCC(=O)Nc1ccc(-c2ncc(-c3ccccc3)s2)cc1] | 132 | C | A | A | NO |
| 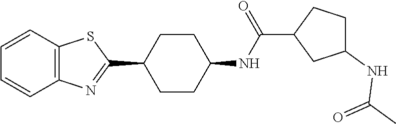 [SMILES: CC(=O)NC1CCC(C(=O)N[C@H]2CC[C@@H](c3nc4ccccc4s3)CC2)C1] | 133 | A | A | A | NO |
| 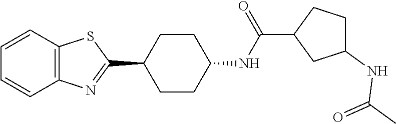 [SMILES: CC(=O)NC1CCC(C(=O)N[C@H]2CC[C@H](c3nc4ccccc4s3)CC2)C1] | 134 | A | A | A | YES |
| 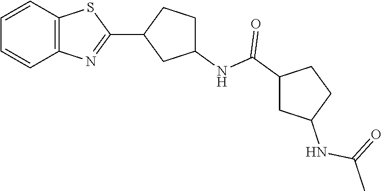 [SMILES: CC(=O)NC1CCC(C(=O)NC2CCC(c3nc4ccccc4s3)C2)C1] | 135 | A | A | A | NO |
| 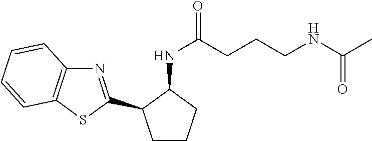 [SMILES: CC(=O)NCCCC(=O)N[C@H]1CCC[C@H]1c1nc2ccccc2s1] | 136 | A | A | A | NO |
| 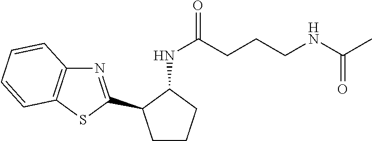 [SMILES: CC(=O)NCCCC(=O)N[C@@H]1CCC[C@H]1c1nc2ccccc2s1] | 137 | A | A | A | NO |
| 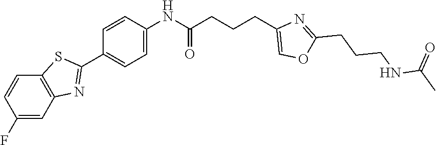 [SMILES: CC(=O)NCCCc1nc(CCCC(=O)Nc2ccc(-c3nc4cc(F)ccc4s3)cc2)co1] | 138 | A | A | B | YES |
| 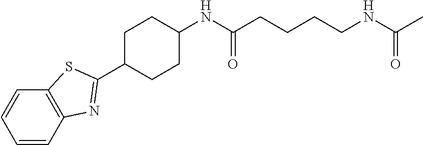 [SMILES: CC(=O)NCCCCC(=O)NC1CCC(c2nc3ccccc3s2)CC1] | 139 | A | A | A | NO |
| 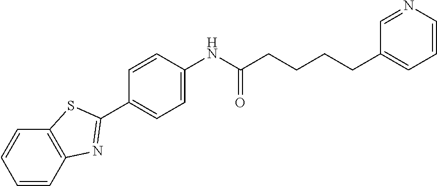 [SMILES: O=C(CCCCc1cccnc1)Nc1ccc(-c2nc3ccccc3s2)cc1] | 140 | C | A | A | YES |
| 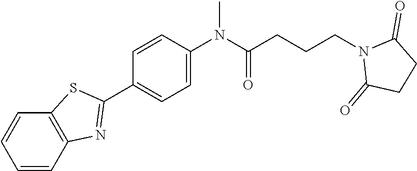 [SMILES: CN(C(=O)CCCN1C(=O)CCC1=O)c1ccc(-c2nc3ccccc3s2)cc1] | 141 | B | A | B | YES |
| 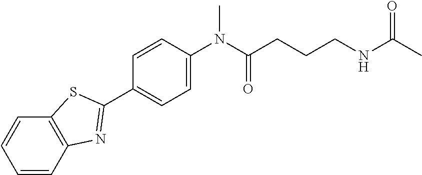 [SMILES: CC(=O)NCCCC(=O)N(C)c1ccc(-c2nc3ccccc3s2)cc1] | 142 | C | A | A | YES |
| 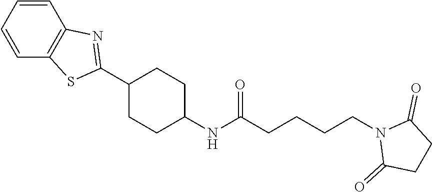 [SMILES: O=C(CCCCN1C(=O)CCC1=O)NC1CCC(c2nc3ccccc3s2)CC1] | 143 | A | A | A | NO |
| 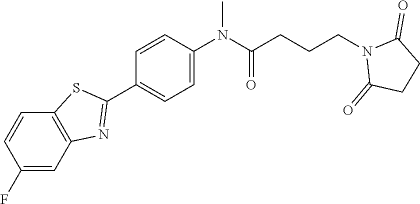 [SMILES: CN(C(=O)CCCN1C(=O)CCC1=O)c1ccc(-c2nc3cc(F)ccc3s2)cc1] | 144 | A | A | A | YES |
| 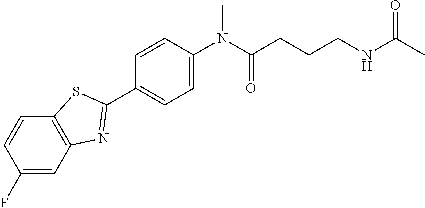 [SMILES: CC(=O)NCCCC(=O)N(C)c1ccc(-c2nc3cc(F)ccc3s2)cc1] | 145 | B | A | A | YES |
| 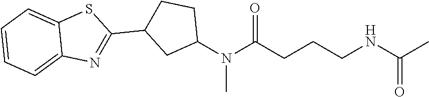 [SMILES: CC(=O)NCCCC(=O)N(C)C1CCC(c2nc3ccccc3s2)C1] | 146 | A | A | A | NO |
| 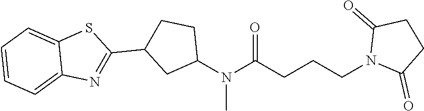 [SMILES: CN(C(=O)CCCN1C(=O)CCC1=O)C1CCC(c2nc3ccccc3s2)C1] | 147 | A | A | A | NO |
| 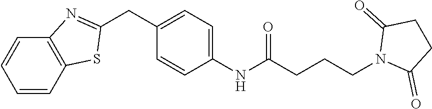 [SMILES: O=C(CCCN1C(=O)CCC1=O)Nc1ccc(Cc2nc3ccccc3s2)cc1] | 148 | A | A | A | NO |
| 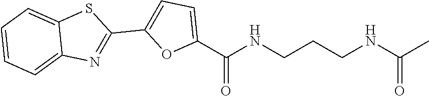 [SMILES: CC(=O)NCCCNC(=O)c1ccc(-c2nc3ccccc3s2)o1] | 149 | A | A | A | YES |
| 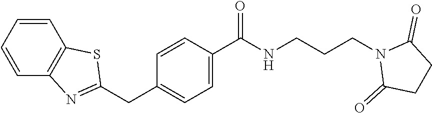 [SMILES: O=C(NCCCN1C(=O)CCC1=O)c1ccc(Cc2nc3ccccc3s2)cc1] | 150 | A | A | A | NO |
| 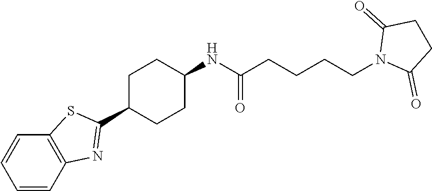 [SMILES: O=C(CCCCN1C(=O)CCC1=O)N[C@H]1CC[C@@H](c2nc3ccccc3s2)CC1] | 151 | A | A | A | YES |
| 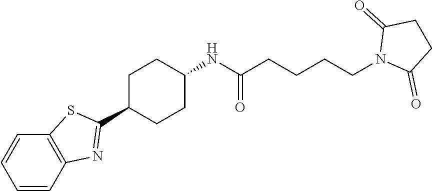 [SMILES: O=C(CCCCN1C(=O)CCC1=O)N[C@H]1CC[C@H](c2nc3ccccc3s2)CC1] | 152 | A | A | A | NO |
| 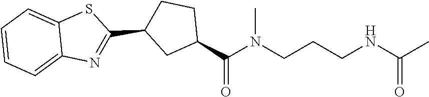 [SMILES: CC(=O)NCCCN(C)C(=O)[C@@H]1CC[C@H](c2nc3ccccc3s2)C1] | 153 | A | A | A | NO |
| 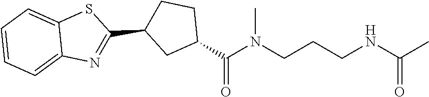 [SMILES: CC(=O)NCCCN(C)C(=O)[C@H]1CC[C@H](c2nc3ccccc3s2)C1] | 154 | A | A | A | NO |
| 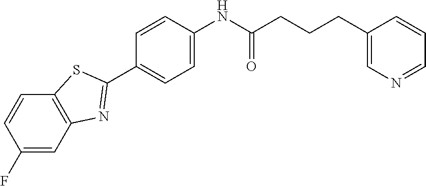 [SMILES: O=C(CCCc1cccnc1)Nc1ccc(-c2nc3cc(F)ccc3s2)cc1] | 155 | C | A | B | YES |
| 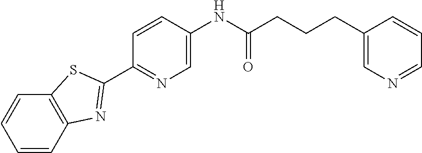 [SMILES: O=C(CCCc1cccnc1)Nc1ccc(-c2nc3ccccc3s2)nc1] | 156 | C | A | A | YES |
| 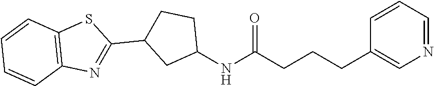 [SMILES: O=C(CCCc1cccnc1)NC1CCC(c2nc3ccccc3s2)C1] | 157 | A | A | A | YES |
| 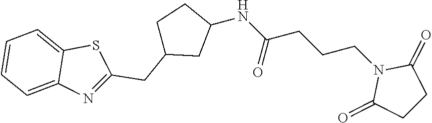 [SMILES: O=C(CCCN1C(=O)CCC1=O)NC1CCC(Cc2nc3ccccc3s2)C1] | 158 | A | A | A | YES |
| 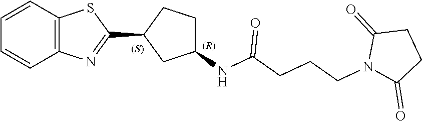 | 159 | A | A | A | NO |
| 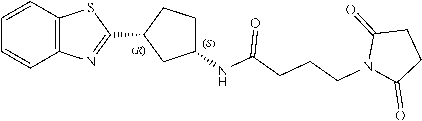 | 160 | A | A | A | NO |
| 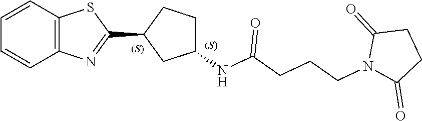 | 161 | A | A | A | YES |
| 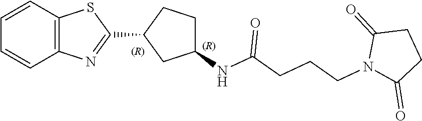 | 162 | A | A | A | YES |
| 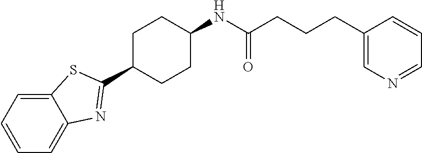 [SMILES: O=C(CCCc1cccnc1)N[C@H]1CC[C@@H](c2nc3ccccc3s2)CC1] | 163 | A | A | A | YES |
| 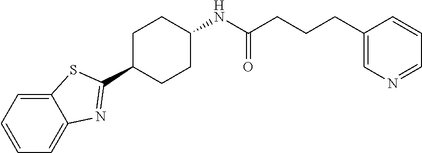 [SMILES: O=C(CCCc1cccnc1)N[C@H]1CC[C@H](c2nc3ccccc3s2)CC1] | 164 | A | A | A | YES |
| 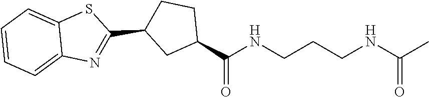 [SMILES: CC(=O)NCCCNC(=O)[C@@H]1CC[C@H](c2nc3ccccc3s2)C1] | 165 | A | A | A | NO |
| 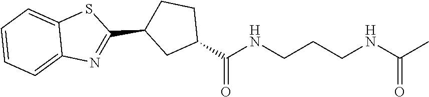 [SMILES: CC(=O)NCCCNC(=O)[C@H]1CC[C@H](c2nc3ccccc3s2)C1] | 166 | A | A | A | NO |
| 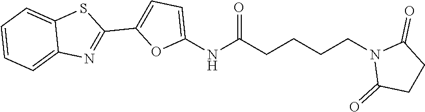 [SMILES: O=C(CCCCN1C(=O)CCC1=O)Nc1ccc(-c2nc3ccccc3s2)o1] | 167 | C | A | A | YES |
| 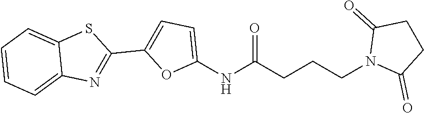 [SMILES: O=C(CCCN1C(=O)CCC1=O)Nc1ccc(-c2nc3ccccc3s2)o1] | 168 | C | A | A | NO |
| 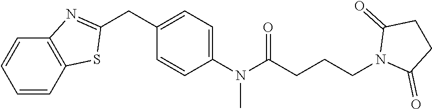 [SMILES: CN(C(=O)CCCN1C(=O)CCC1=O)c1ccc(Cc2nc3ccccc3s2)cc1] | 169 | A | A | A | YES |
| 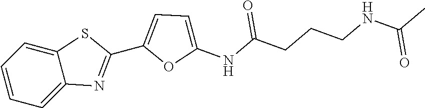 [SMILES: CC(=O)NCCCC(=O)Nc1ccc(-c2nc3ccccc3s2)o1] | 170 | B | A | A | YES |
| 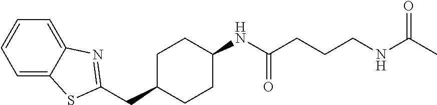 [SMILES: CC(=O)NCCCC(=O)N[C@H]1CC[C@@H](Cc2nc3ccccc3s2)CC1] | 171 | A | A | A | YES |
| 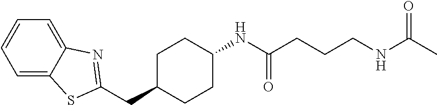 [SMILES: CC(=O)NCCCC(=O)N[C@H]1CC[C@H](Cc2nc3ccccc3s2)CC1] | 172 | A | A | A | YES |
| 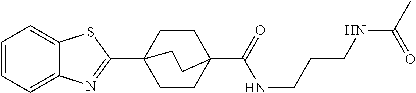 [SMILES: CC(=O)NCCCNC(=O)C12CCC(c3nc4ccccc4s3)(CC1)CC2] | 173 | A | A | A | YES |
| 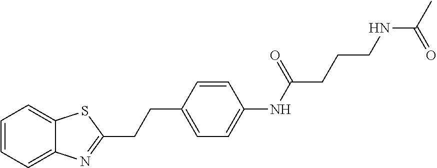 [SMILES: CC(=O)NCCCC(=O)Nc1ccc(CCc2nc3ccccc3s2)cc1] | 174 | B | A | A | YES |
| 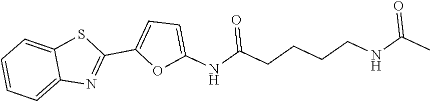 [SMILES: CC(=O)NCCCCC(=O)Nc1ccc(-c2nc3ccccc3s2)o1] | 175 | C | A | A | YES |
| 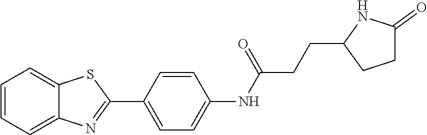 [SMILES: O=C(CCC1CCC(=O)N1)Nc1ccc(-c2nc3ccccc3s2)cc1] | 176 | B | B | A | NO |
| 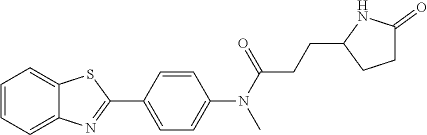 [SMILES: CN(C(=O)CCC1CCC(=O)N1)c1ccc(-c2nc3ccccc3s2)cc1] | 177 | A | A | A | YES |
| 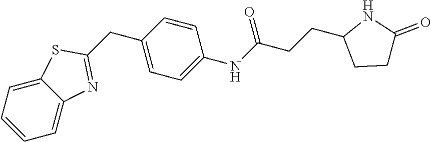 [SMILES: O=C(CCC1CCC(=O)N1)Nc1ccc(Cc2nc3ccccc3s2)cc1] | 178 | A | A | A | YES |
| 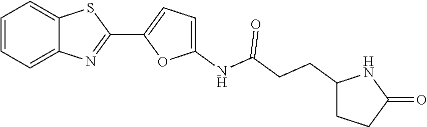 [SMILES: O=C(CCC1CCC(=O)N1)Nc1ccc(-c2nc3ccccc3s2)o1] | 179 | A | A | A | YES |
| 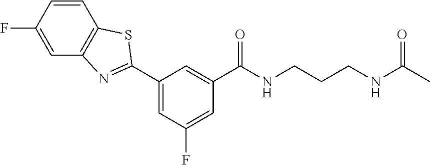 [SMILES: CC(=O)NCCCNC(=O)c1cc(F)cc(-c2nc3cc(F)ccc3s2)c1] | 180 | A | A | A | NO |
| 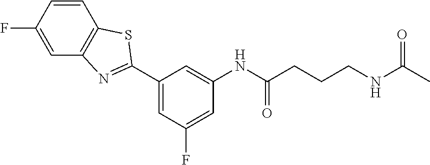 [SMILES: CC(=O)NCCCC(=O)Nc1cc(F)cc(-c2nc3cc(F)ccc3s2)c1] | 181 | A | A | A | NO |
| 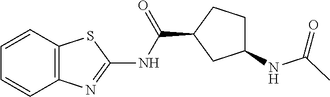 [SMILES: CC(=O)N[C@@H]1CC[C@H](C(=O)Nc2nc3ccccc3s2)C1] | 182 | A | A | A | YES |
| 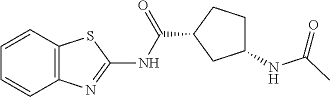 [SMILES: CC(=O)N[C@H]1CC[C@@H](C(=O)Nc2nc3ccccc3s2)C1] | 183 | A | A | A | YES |
| 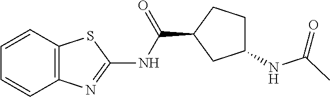 [SMILES: CC(=O)N[C@H]1CC[C@H](C(=O)Nc2nc3ccccc3s2)C1] | 184 | B | A | A | YES |
| 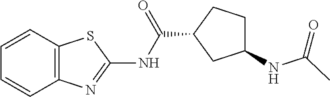 [SMILES: CC(=O)N[C@@H]1CC[C@@H](C(=O)Nc2nc3ccccc3s2)C1] | 185 | A | A | A | YES |
| 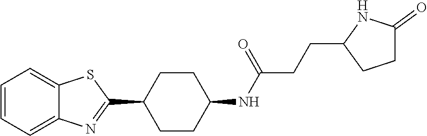 [SMILES: O=C1CCC(CCC(=O)N[C@H]2CC[C@@H](c3nc4ccccc4s3)CC2)N1] | 186 | A | A | A | NO |
| 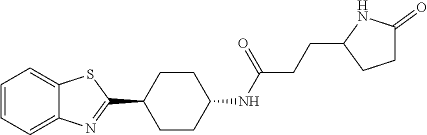 [SMILES: O=C1CCC(CCC(=O)N[C@H]2CC[C@H](c3nc4ccccc4s3)CC2)N1] | 187 | A | A | A | NO |
| 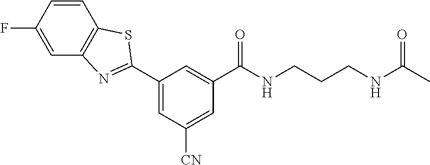 [SMILES: CC(=O)NCCCNC(=O)c1cc(C#N)cc(-c2nc3cc(F)ccc3s2)c1] | 188 | A | A | A | YES |

Keys: A = >10 mM; B = 1 to 10 mM; C = <1 mM

### Example 7

Protocol for RNA Isolation and qPCR to Determine MBD3L2 RNA Levels

The protocol describes the process of isolating RNA from cultured cells, converting the RNA to complementary DNA (cDNA), and measuring target gene expression using a quantitative polymerase chain reaction (qPCR).

Treatment of Cells with Compound

The seed FSHD cells were dispensed into a six-well dish. One day after seeding, the DUX4 inhibitor (11 μM or 3.6 μM/well) was added to the cells. After another 48 hrs, the old media was removed from the cells. The cells were washed with 2 mL of warm phosphate buffered saline (PBS).

The PBS was aspirated, and the cells were lysed in each well with 350 μL of RLT buffer+3.5 μL of beta-mercaptoethanol (BME). The lysates were added to fresh, RNAse-free, labeled Eppendorf tubes. The cells were then physically disrupted for 60 min at level 4 of the bead disrupter to ensure the release of the RNA.

### Collection of RNA

The RNA was collected according to standard procedures (i.e., Qiagen kit instructions) as discussed below.

The RNA was precipitated by addition of 350 μl of 70% ethanol to each sample in a separate Eppendorf tube. Each suspension was mixed by pipetting up and down.

The solution was transferred into the pink spin column (700 μL). Up to 700 μL of the sample, including any precipitate that formed, was transferred to an RNeasy spin column placed in a 2 mL collection tube. The tube was centrifuged for 15 s at 13K RPMS, and the flowthrough was discarded.

Buffer RW1 (700 μL) was added to the RNeasy spin column. The tube was centrifuged for 15 s at 13K RPMS, and the flowthrough was discarded.

Buffer RPE (500 μL) was added to the RNeasy spin column. The tube was centrifuged for 15 s at 13K RPMS, and the flowthrough was discarded.

Additional Buffer RPE (500 μL) was added to the RNeasy spin column. The tube was centrifuged for 15 s at 13K RPMS, and the flowthrough was discarded.

The column was placed in a fresh collecting tube and centrifuged for 2 min at 13K RPMS.

The RNA was then eluted and collected from the column. Water (30 μL) was added in the center of the column and allowed to incubate for 5 min at rt. The column was then centrifuged for 1 min at 14K RPM, after which the RNA concentration was measured.

### RNA Concentration Measurement

The nanodrop measurement was calibrated with 2 μL of nuclease-free water as a blank. The RNA sample (2 μL) was added to the nanodrop, and the RNA concentration was measured. Once the concentration was determined, the samples were prepared for reverse transcription.

### Reverse Transcription

The RNA concentrations were standardized to 200 μg/μL. In a PCR tube, RNA (2 μg) in a new Eppendorf tube was diluted with RNase-free water to a 9.5 μL final volume of 200 μg/μL RNA.

DNase was used to remove any DNA contamination. A DNase Master Mix was prepared from 1.5 μL/reaction DNase solution (1 unit/μL, Promega), 3 μL/reaction 5× RT buffer (i.e., 250 mM Tris-HCl (pH 8.3), 375 mM KCl, 15 mM MgCl₂, and 500 μl 0.1 M DTT; Promega MMLV), and 1 μL/reaction RNase inhibitor solution (RNasin, 40 units/μL, Promega). The DNase Master Mix was added to the standardized RNA samples (5.5 μL/sample). The samples were mixed by agitation and briefly spun down by centrifuge. The samples were heated to 37° C. for 60 min, 80° C. for 5 min, and then cooled to 4° C. The samples were stored on ice until the addition of the next reagents.

An RT Reaction Master Mix was prepared from 5 μL/reaction 5× RT buffer, 2 μL/reaction dNTP solution (2.5 mM in each nucleotide), 1 μL/reaction RNase inhibitor solution (RNasin, 40 units/μL, Promega), 1.6 μL/reaction M-MLV reverse transcriptase (200 units/μL, Promega), and 13.4 μL/reaction deionized, RNase-free water. The RT Reaction Master Mix was cooled on ice until use.

A 50 μM solution of Random Primer 6 random hexanucleotides (2 μL) was added to each sample. The samples were mixed by agitation and briefly spun down by centrifuge. The samples were heated to 70° C. for 5 min and then cooled to 4° C.

The RT Reaction Master Mix (23 μL) was added to each sample. The samples were mixed by agitation and briefly spun down by centrifuge. To convert the RNA to cDNA, the samples were heated to 42° C. for 60 min, 95° C. for 5 min, and then cooled to 4° C. The cDNA product mixture was diluted 5:1 with deionized water (40 μL+160 μL).

Running the qPCR

The samples were run in triplicate, with a methyl-CpG-binding protein 3-like 2 (MBD3L2) target primer and a eukaryotic translation elongation factor 1-alpha (EEF1A) control primer.

An qPCR Master Mix was prepared for each primer and stored on ice (4° C.) until use. The qPCR Master Mix included 10 μL/reaction SYBR Green Mix (2×) (ThermoFisher), 1 μL/reaction of the primer mix (i.e., the MBD3L2 PCR Primer mix or the EEF1A Primer mix), and 4 μL/reaction deionized, RNase-free water.

To set up the qPCR plate, 15 μL of the appropriate qPCR Master Mix was added to the appropriate wells for each target/primer. After all the targets/primers have been added to the wells, 5 μL of the sample was added to the corresponding wells so that each well is 20 μL total. The plate was covered with a clear sheet.

An qPCR run was set up (QuantStudio 5) with the following cycle:

$$Hold stage ( 1 cycle ) : 50 ° C - 2 min ( 1.6 ° C / s ) ; 95 ° C - 10 min ( 1.6 ° C / s ) .$$

$$PCR stage ( 50 cycles ) : 95 ° C - 15 sec ( 1.6 ° C / s ) for denaturing ; 60 ° C - 1 min ( 1.6 ° C / s ) for amplification .$$

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

What is claimed:

1. A compound of Formula I: 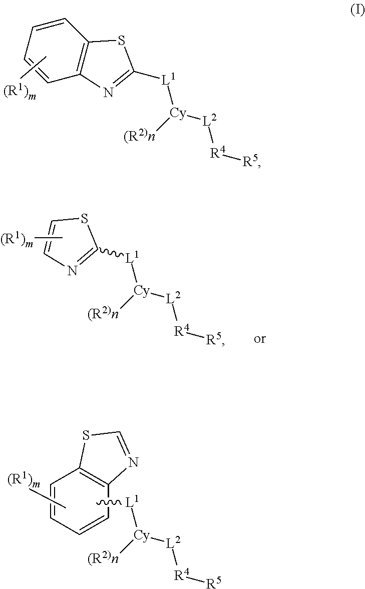
or a pharmaceutically acceptable salt thereof;
wherein:
each R¹ is independently selected from the group consisting of H and R²;
m is an integer from 0 to 4;
a wavy bond indicates a single bond attachment from L to a free site of a thiazole or benzothiazole ring,
L¹ is selected from the group including a single bond, C₁₋₆alkyl, and —(C═O)—;
L² is selected from the group including —(C═O)(NR³)—, —(C═O)-alkyl-, and (NR³)(C═O)—;
Cy is selected from the group consisting of C₃₋₉cycloalkyl, C₃₋₉heterocycyl, C₃-C₉heteroaryl, and C₆₋₁₀aryl;
each R² is independently selected from the group consisting of halo, C₁₋₃alkoxy, C₁₋₃alkyl, cyano, and R⁵;
n is an integer from 0 to 2;
each R³ is independently selected from the group consisting of H and C₁₋₃alkyl;
R⁴ is C₁₋₆alkylene, wherein R⁴ is substituted with from 0 to 4 R⁷ groups;
R⁵ is selected from the group consisting of —O(CO)R⁶, —N(R³)(CO)R³, —N(R³)(CO)R⁶, —OR⁶, —(CO)R⁶, —(CO)N(R³)R³, —(CO)N(R³)R⁶, C₃₋₇cycloalkyl, C₃₋₉heterocycyl, C₆₋₁₀aryl, and C₃-C₉heteroaryl;
or, alternatively, R⁴ and R⁵ join to form a 5- to 8-membered cycloalkyl or heterocyclic ring that is optionally substituted with from 0 to 4 R⁷ groups;
each R⁶ is independently selected from the group consisting of C₁₋₆alkyl, C₃₋₇cycloalkyl, C₃₋₉heterocycyl, C₆₋₁₀aryl, and C₃-C₉heteroaryl; wherein R⁶ is substituted with from 0 to 4 R⁷ groups; and
each R⁷ is independently selected from the group consisting of halo, C₁₋₃alkoxy, and C₁₋₃alkyl.

2. The compound of claim 1, wherein the compound is of Formula II: 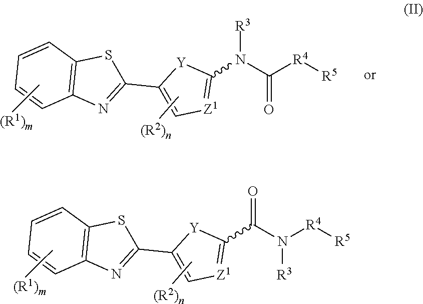
or a pharmaceutically acceptable salt thereof;
wherein:
Y is selected from the group consisting of CH, CR², —N═CH—, —N═CR²—, O, S, and N;
wherein Y and Z¹ are not both N; and
Z¹ is selected from the group consisting of CH, CR², —N═CH—, —N═CR²—, and N;
wherein Y and Z¹ are not both N.

3. The compound of claim 1, wherein the compound is of Formula III: 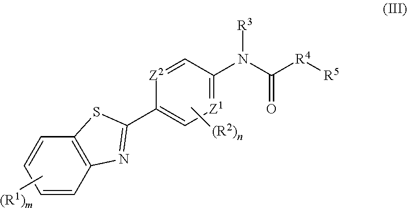
or a pharmaceutically acceptable salt thereof;
wherein:
Z¹ and Z² are each selected from the group consisting of CH, CR², and N; wherein Z¹ and Z² are not both N.

4. The compound of claim 3, wherein the compound is of Formula IV: 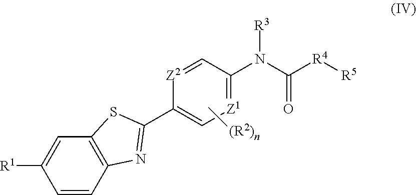
or a pharmaceutically acceptable salt thereof;
wherein:
R¹ is selected from the group consisting of H, halo, and C₁₋₃alkyl;
each R² is independently selected from the group consisting of halo, C₁₋₃alkoxy, and C₁₋₃alkyl;
R⁴ is C₁₋₆alkylene;
R⁵ is selected from the group consisting of —O(CO)R⁶, —NH(CO)R⁶, —OR⁶, —(CO)R⁶, C₃₋₇cycloalkyl, and C₃₋₉heterocycyl; and
R⁶ is selected from the group consisting of C₁₋₆alkyl, C₃₋₇cycloalkyl, C₃₋₉heterocyclyl, and C₃₋₉heteroaryl.

5. The compound of claim 3 or 4, wherein Z¹ and Z² are each selected from the group consisting of CH and N.

6. The compound of any one of claims 1-5, wherein R¹ is H or methyl.

7. The compound of any one of claims 1-6, wherein n is 0.

8. The compound of any one of claims 1-7, wherein R⁴ is substituted with 0 R² groups.

9. The compound of any one of claims 1-8, wherein R⁵ is selected from the group consisting of —NH(CO)CH₃, —O(CO)CH₃, —(CO)CH₃, and —OCH₂CH₃.

10. The compound of claim 1, wherein the compound is selected from the group consisting of:

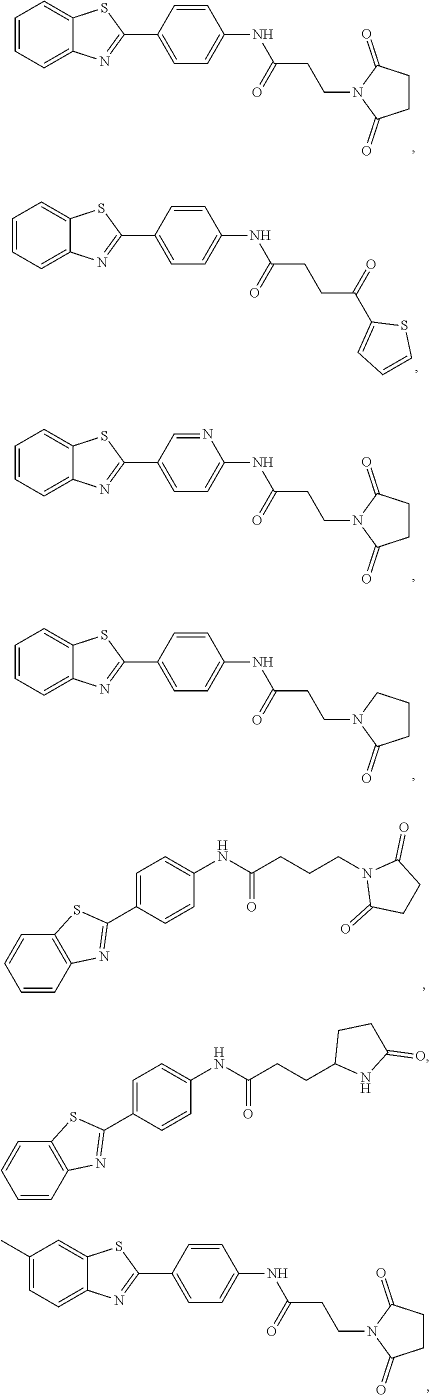

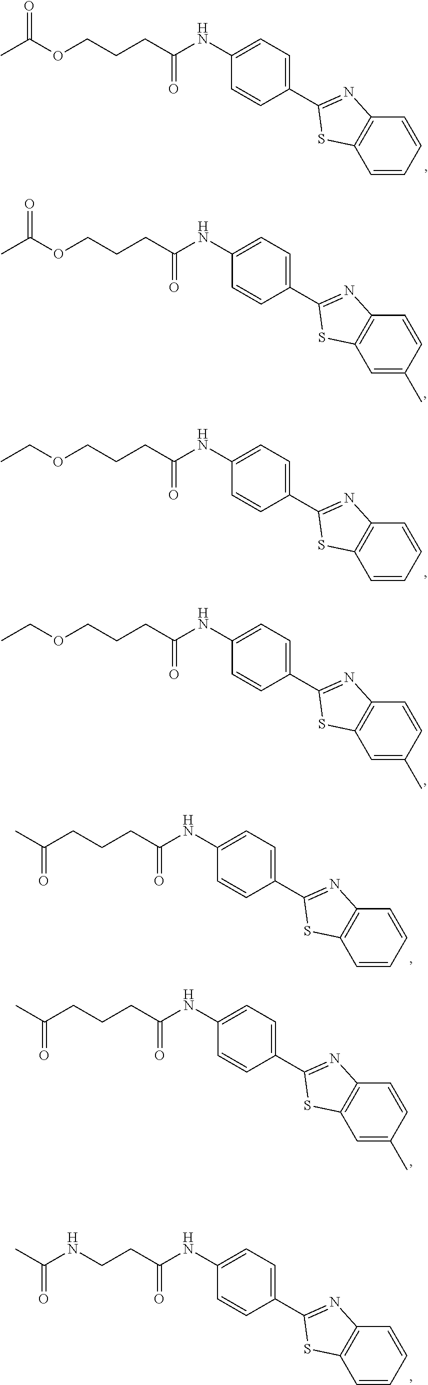

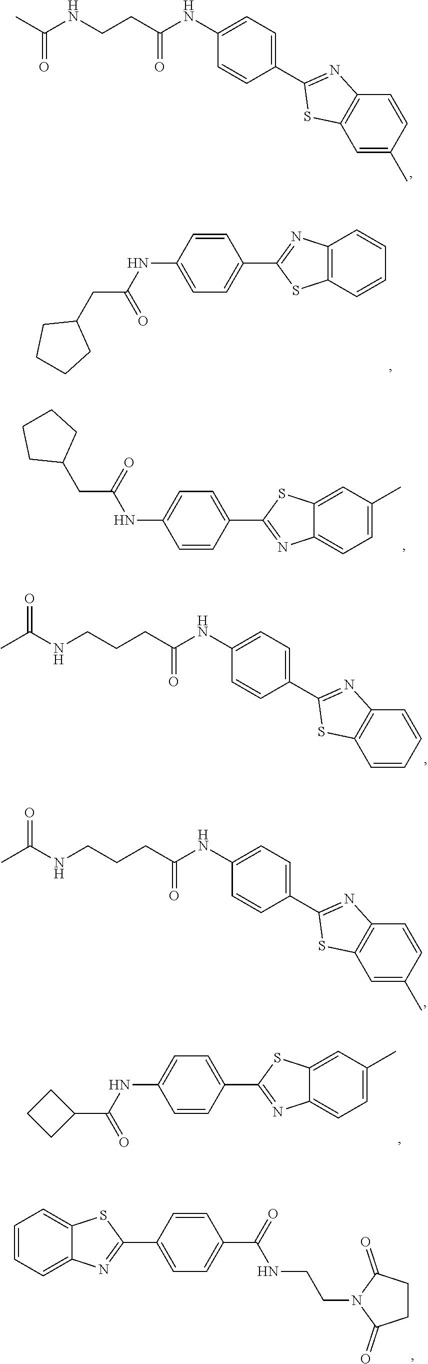

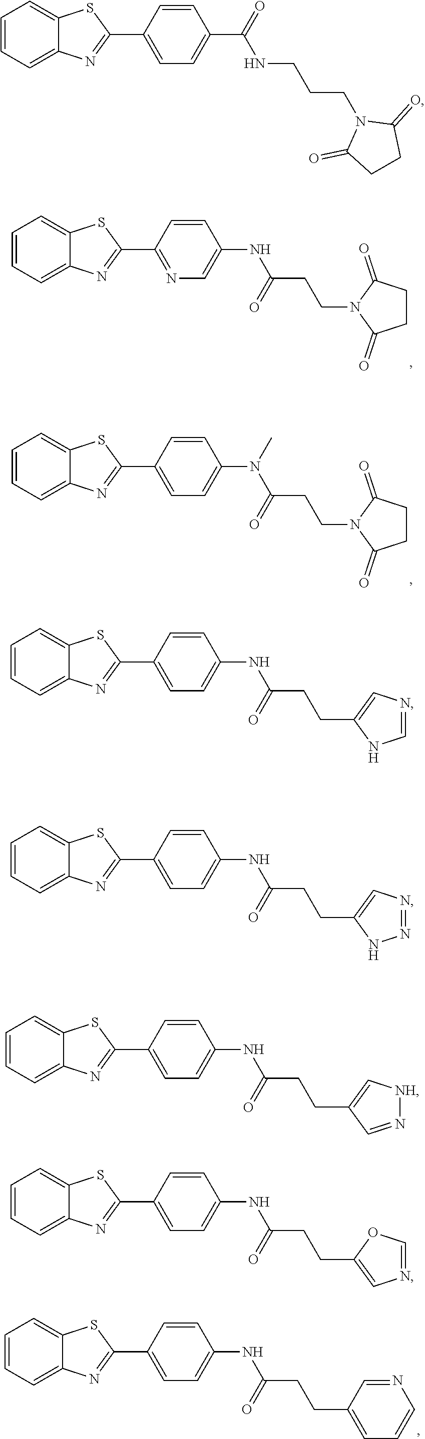

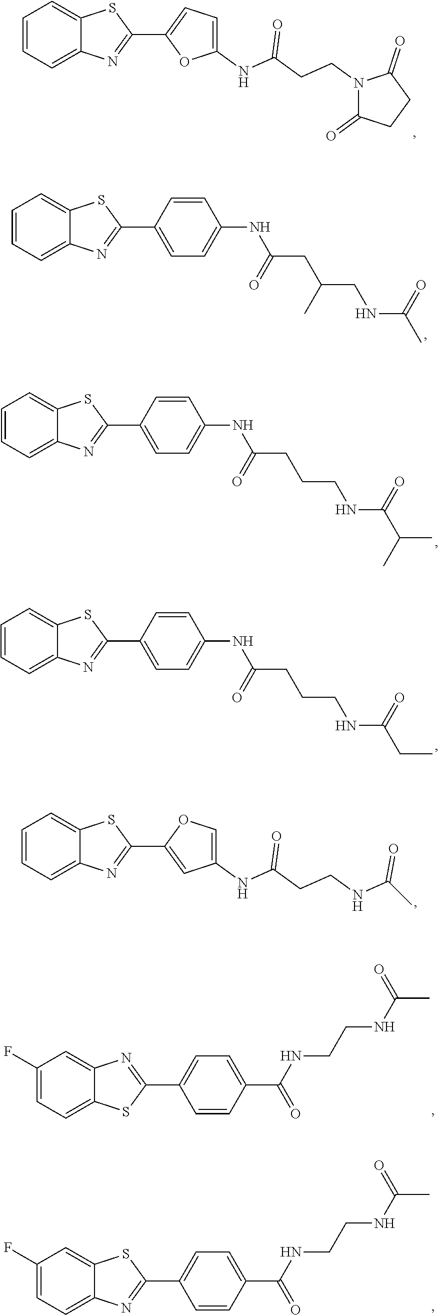

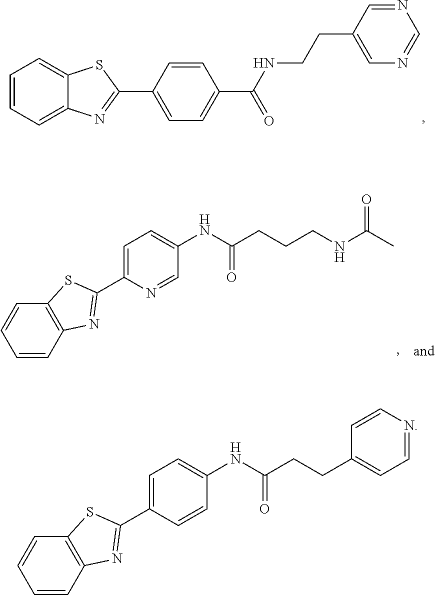 [SMILES: O=C(NCCc1cncnc1)c1ccc(-c2nc3ccccc3s2)cc1 ; CC(=O)NCCCC(=O)Nc1ccc(-c2nc3ccccc3s2)nc1 ; O=C(CCc1ccncc1)Nc1ccc(-c2nc3ccccc3s2)cc1]

11. The compound of claim 10, wherein the compound is selected from the group consisting of:

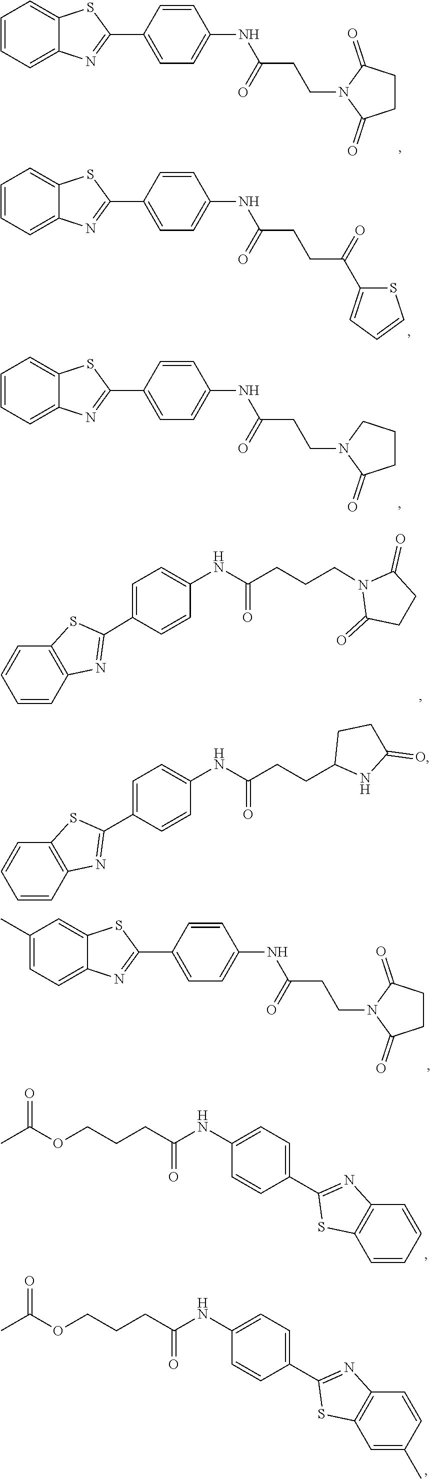

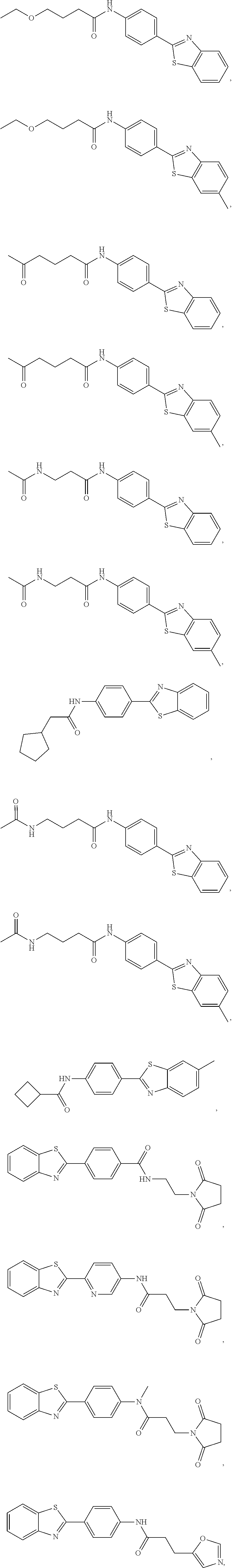

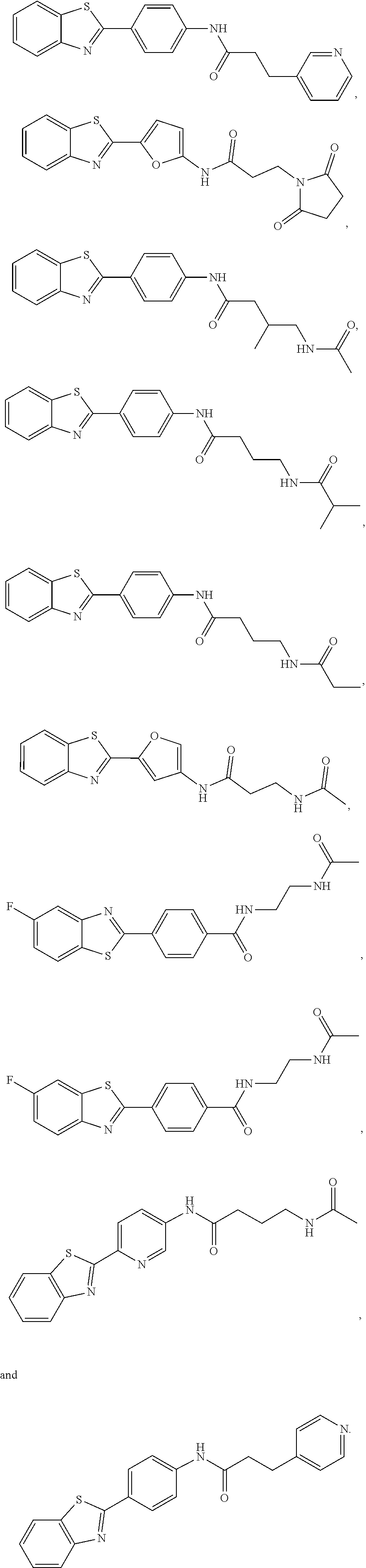

12. The compound of claim 1, wherein the compound is selected from the group consisting of the compounds of Table 6-1.

13. The compound of claim 12, wherein the compound inhibits the production of MBD3L2 RNA at 11 mM.

14. The compound of claim 12 or 13, wherein the compound has a DUX4 EC₅₀ of <10 mM.

15. The compound of claim 14, wherein the compound has a DUX4 EC₅₀ of <1 mM.

16. A pharmaceutical composition comprising:
the compound of any one of claims 1-15, and
a pharmaceutically acceptable excipient, carrier or diluent.

17. The pharmaceutical composition of claim 16, wherein the composition is an oral formulation.

18. A method for the treatment of a patient comprising the administration of an effective treatment amount of a compound or composition of any one of claims 1-17.

19. The method of claim 18, wherein the host is a human.
